(19) United States
(12) Patent Application Publication
(10) Pub. No.: US 2015/0284360 A1
(43) Pub. Date: Oct. 8, 2015

(54) AMIDE DERIVATIVES OF ANILINE-RELATED COMPOUNDS AND COMPOSITIONS THEREOF

(71) Applicant: C&C BIOPHARMA, LLC, Los Angeles, CA (US)

(72) Inventors: Lin CHEN, La Canada, CA (US); Yongqing WU, Irvine, CA (US); Dahai GAI, La Crescenta, CA (US); Xiaojiang CHEN, South Pasadena, CA (US)

(21) Appl. No.: 14/677,753

(22) Filed: Apr. 2, 2015

(30) Foreign Application Priority Data
Apr. 2, 2014 (CN) 201410130960.X

(51) Int. Cl. C07D 401/04; C07C 235/74; C07D 213/56

(52) U.S. Cl. CPC C07D 401/04; C07D 213/56; C07C 235/74

(57) ABSTRACT

Amide derivatives of aniline-related compounds are disclosed, including salts thereof, and compositions, preparations and uses thereof. In certain embodiments, the amide derivatives and/or salts thereof show higher solubility in water compared to the corresponding parent aniline-related compounds.

### PRIORITY CLAIM

The present application claims priority to Chinese Application No. 201410130960.X, filed Apr. 2, 2014, the disclosure of which is incorporated by reference herein in its entirety.

### BACKGROUND

Compounds comprising an aniline structure (aniline-related compounds) are promising drug candidates for various treatments. However, aniline-related compounds tend to have low solubility in water, which limits their bioavailability. Thus, there exists a need for novel derivatives thereof as with higher water solubility.

### SUMMARY

One aspect of the invention relates to amide derivatives of aniline-related compounds having improved solubility.

Another aspect of the invention relates to compositions of the amide derivatives disclosed herein.

Another aspect of the invention relates to uses of the amide derivatives disclosed herein.

### BRIEF DESCRIPTION OF THE DRAWINGS

FIG. 1. Structures of acids (optionally protected) used to modify aniline-related compounds.

FIG. 2. Structures of several protected MS-275 amide derivatives.

FIG. 3. Structures of several MS-275 amide derivatives and salts thereof.

FIG. 4. Structures of several protected MGCD0103 amide derivatives.

FIG. 5. Structures of several MGCD0103 amide derivatives and salts thereof.

FIG. 6. Structures of several protected PAOA amide derivatives.

FIG. 7. Structures of several PAOA amide derivatives and salts thereof.

FIG. 8. Structures of several protected CC30 amide derivatives.

FIG. 9. Structures of several CC30 amide derivatives and salts thereof.

FIG. 10. Conversion of the prodrug Lys-CC30.2HCl to the parent drug CC30 in vitro and in vivo. (A) In vitro conversion of Lys-CC30.2HCl to the parent drug CC30 in rat plasma. (B) In vivo conversion of Lys-CC30.2HCl to the parent drug CC30 in the rat.

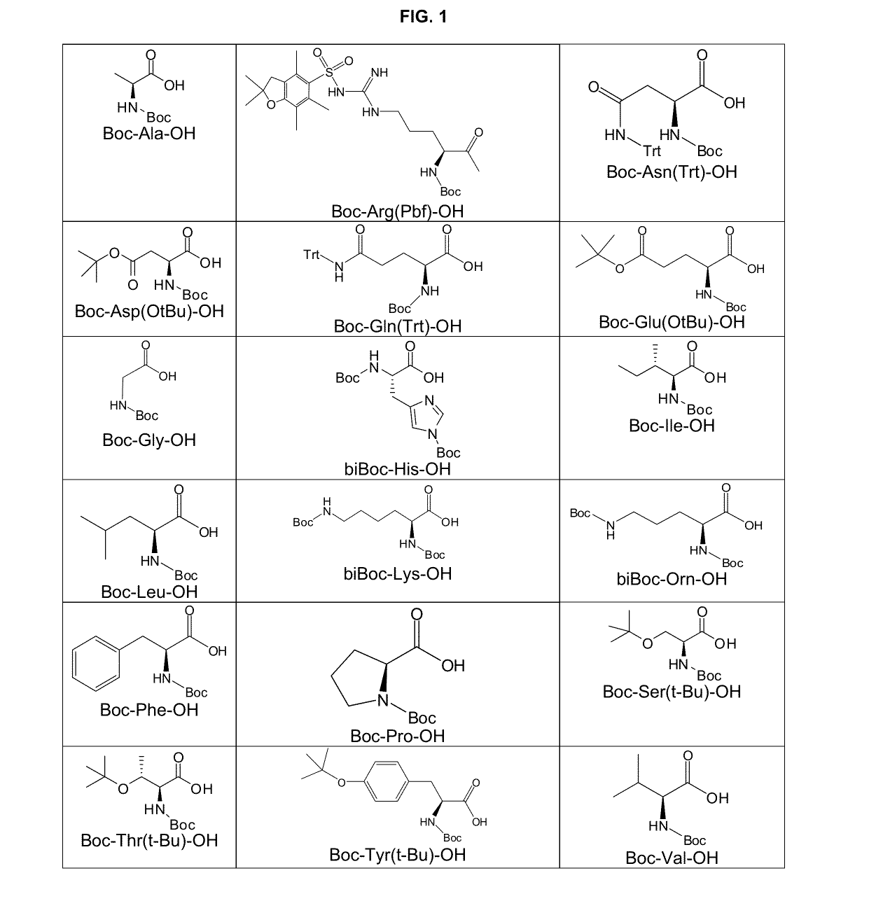

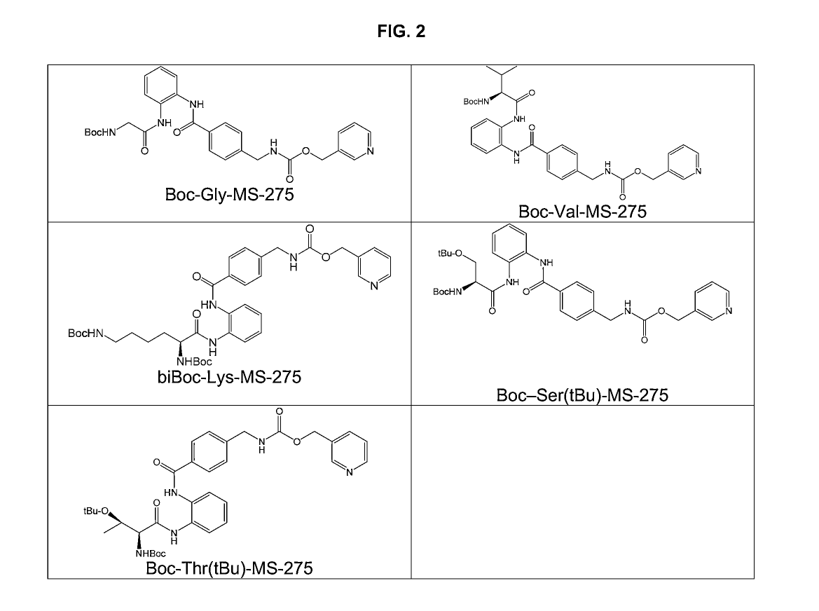

### DETAILED DESCRIPTION

Novel amide derivatives of aniline-related compounds are disclosed herein. Such amide derivatives have shown improved solubility. The amide derivatives and/or compositions thereof may be used for at least the purposes for which the aniline-related compounds are used.

As used herein, an “amide derivative,” an “amide derivative of an aniline-related compound,” an “aniline-related compound amide derivative” are used interchangeably. An amide derivative also includes crystals thereof, stereoisomers thereof, pharmaceutically acceptable solvates thereof, pharmaceutically acceptable salts thereof, an any mixtures thereof in any ratio.

As used herein, an “aniline-related compound” means a compound comprising an amino aryl group as defined below (e.g. phenyl) or an amino heteroaryl group as defined below (e.g. pyridinyl).

I. Compounds

One aspect of the invention relates to a compound comprising a structure of Structure X:

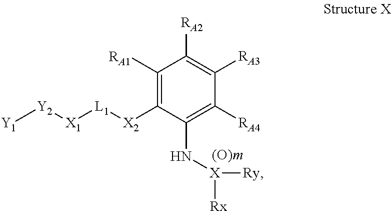

including crystals, stereoisomers, pharmaceutically acceptable solvates, and pharmaceutically acceptable salts thereof, further including mixtures thereof in all ratios, wherein:

each R_{A1}-R_{A4} are independently selected from the group consisting of H, F, Cl, Br, and I;

X₂ is selected from the group consisting of —C(═O)—NH— and —NH—C(═O)—;

L₁ is —(CH₂)ₙ—, wherein n is 1, 2, 3, 4, 5, 6, 7, 8, 9, or 10, and any one or more of the —CH₂— may be replaced by a group selected from the group consisting of aryl (e.g. phenylene, 1,4-phenylene), heteroaryl, cycloalkyl (e.g. cyclohexylene, 1,4-cyclohexylene), heterocycloalkyl, substituted aryl, substituted heteroaryl, substituted cycloalkyl, substituted heterocycloalkyl, —O—, —S—, —C(═O)—, —S(═O)—, —S(═O)₂—, —NH—C(═O)—, —C(═O)—NH—, —NR— (wherein R is hydrogen, alkyl or aryl), —C═C—, and —C≡C—;

X₁ is selected from the group consisting of —C(═O)—NH—, —NH—C(═O)—, and —NH—;

Y₁ is selected from the group consisting of H, aryl, heteroaryl, cycloalkyl, heterocycloalkyl, substituted aryl, substituted heteroaryl, substituted cycloalkyl, substituted heterocycloalkyl, —OH, —SH, and —NH₂;

Y₂ is —(CH₂)ₚ—, wherein p is 1, 2, 3, 4, 5, 6, 7, 8, 9, or 10, and any one or more of the —CH₂— may be replaced by a group selected from the group consisting of alkyl, —C═C—, —C≡C—, aryl, heteroaryl (e.g. 2,4-pyrimidylene), cycloalkyl, heterocycloalkyl, substituted aryl, substituted heteroaryl, substituted cycloalkyl, substituted heterocycloalkyl, —O—, —S—, —N—, —C(═O)—, and —C(═S)—;

X is S, P or C, wherein:

when X is S, and m is 2, Rx is selected from the group consisting of H, alkyl, aryl, heteroaryl, cycloalkyl, heterocycloalkyl, substituted alkyl, substituted aryl, substituted heteroaryl, substituted cycloalkyl, and substituted heterocycloalkyl; and Ry is nothing;

when X is P, and m is 1, Rx and Ry are independently selected from the group consisting of H, alkyl, aryl, heteroaryl, cycloalkyl, heterocycloalkyl, substituted alkyl, substituted aryl, substituted heteroaryl, substituted cycloalkyl, and substituted heterocycloalkyl;

when X is C, and m is 0, Rx is H, and Ry is

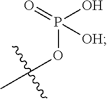 [SMILES: *OP(=O)(O)O]

and
when X is C, and m is 1, Rx is nothing, and Ry is alkyl, alkyl carboxyl,

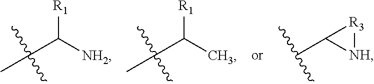

wherein:
R₁ selected from the group consisting of H,

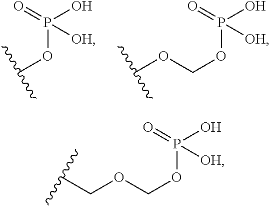 [SMILES: *OP(=O)(O)O ; *OCOP(=O)(O)O ; *COCOP(=O)(O)O]

alkyl, and alkyl further substituted with aryl, heteroaryl, amino, hydroxide, hydroxide aryl, hydroxide carbonyl, amino carbonyl, thiol, alkyl-S—, or guanidinyl; and
R3 is alkyl.

As used herein, unless otherwise specified, the substitution groups having the same names are defined the same as supra.

As used herein, unless otherwise specified, the compound comprising a structure of Structure X has more desirable properties (e.g. higher water solubility) than a corresponding parent compound comprising a structure of Structure PR:

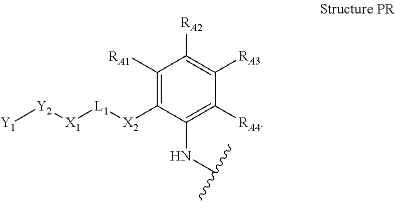

In certain embodiments, the corresponding parent drug comprising a structure of Structure PR has a structure of Structure PH:

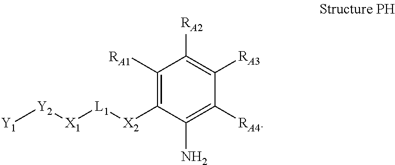

In certain embodiments, when Structure PR is Structure PR-1, X is C, and m is 0, Rx and Ry are not both H.

For example, in an embodiment, a compound comprising a structure of Structure X as defined above, including crystals, stereoisomers, pharmaceutically acceptable solvates, and pharmaceutically acceptable salts thereof, further including mixtures thereof in all ratios, wherein:

X, m, Rx and Ry are defined the same as supra; and

Structure PR is selected from the group consisting of Structure PR-1, Structure PR-2, Structure PR-3, Structure PR-4, Structure PR-5, Structure PR-6, Structure PR-7, Structure PR-8, and Structure PR-9 shown in Table 1:

TABLE 1
Structures of Structures PR-1~PR-9

| Name of Structure PR | 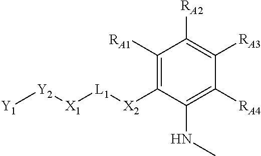 | Parent compound (Structure PH) |
|---|---|---|
| Structure PR-1 | 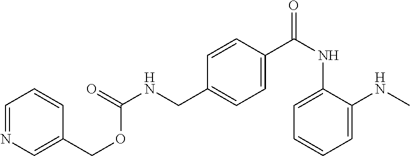 [SMILES: CNc1ccccc1NC(=O)c1ccc(CNC(=O)OCc2cccnc2)cc1] | Entinostat (MS-275) |
| Structure PR-2 | 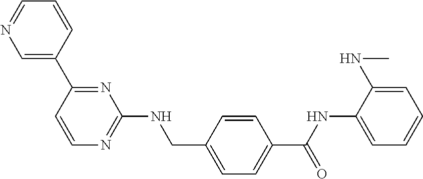 [SMILES: CNc1ccccc1NC(=O)c1ccc(CNc2nccc(-c3cccnc3)n2)cc1] | Mocetinostat (MGCD0103) |
| Structure PR-3 | 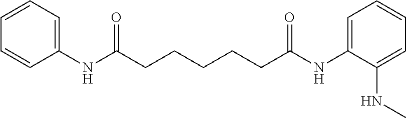 [SMILES: CNc1ccccc1NC(=O)CCCCCC(=O)Nc1ccccc1] | PAOA |
| Structure PR-4 | 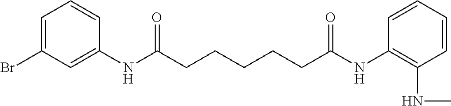 [SMILES: CNc1ccccc1NC(=O)CCCCCC(=O)Nc1cccc(Br)c1] | CC30 |
| Structure PR-5 | 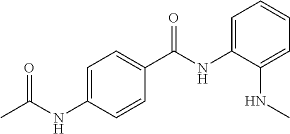 [SMILES: CNc1ccccc1NC(=O)c1ccc(NC(C)=O)cc1] | Acetyldinaline (CI 994) |
| Structure PR-6 | 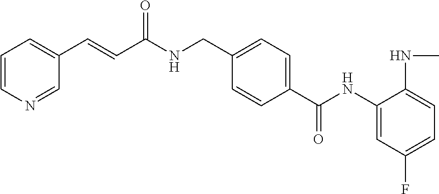 [SMILES: CNc1ccc(F)cc1NC(=O)c1ccc(CNC(=O)/C=C/c2cccnc2)cc1] | Chidamide |
| Structure PR-7 | 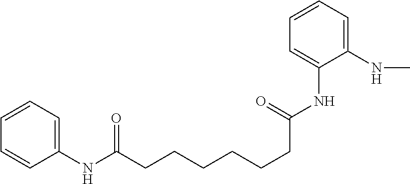 [SMILES: CNc1ccccc1NC(=O)CCCCCCC(=O)Nc1ccccc1] | BML-210 |
| Structure PR-8 | 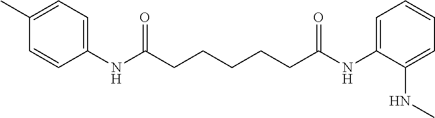 | Pimelic Diphenylamide 106 |
| Structure PR-9 | 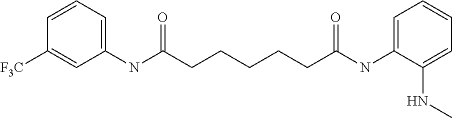 [SMILES: CNc1ccccc1NC(=O)CCCCCC(=O)Nc1cccc(C(F)(F)F)c1] | CC54 |

In another embodiment, Structure PR is selected from the group consisting of Structures PR-1, Structure PR-2, Structure PR-3, Structure PR-4, Structure PR-5, Structure PR-6, Structure PR-7, Structure PR-8, and Structure PR-9; X is C; m is 1; Rx is nothing; and Ry is an alkyl or alkyl carboxyl.

In another embodiment, Structure PR is selected from the group consisting of Structures PR-7 and PR-3; X is C; m is 1; Rx is nothing; and Ry is an alkyl or alkyl carboxyl.

In another embodiment, Structure PR is Structure PR-4; X is C; m is 1; Rx is nothing; and Ry is an alkyl or alkyl carboxyl.

In another embodiment, Structure PR is selected from the group consisting of Structures PR-1, Structure PR-2, Structure PR-3, Structure PR-4, Structure PR-5, Structure PR-6, Structure PR-7, Structure PR-8, and Structure PR-9; X is C; m is 1; Rx is nothing; Ry is

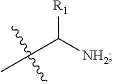

R₁ is selected from the group consisting of H, alkyl, and alkyl further substituted with a substituent selected from the group consisting of aryl, heteroaryl, amino, hydroxide, hydroxide aryl, hydroxide carbonyl, amino carbonyl, thiol, alkyl-S—, and guanidinyl.

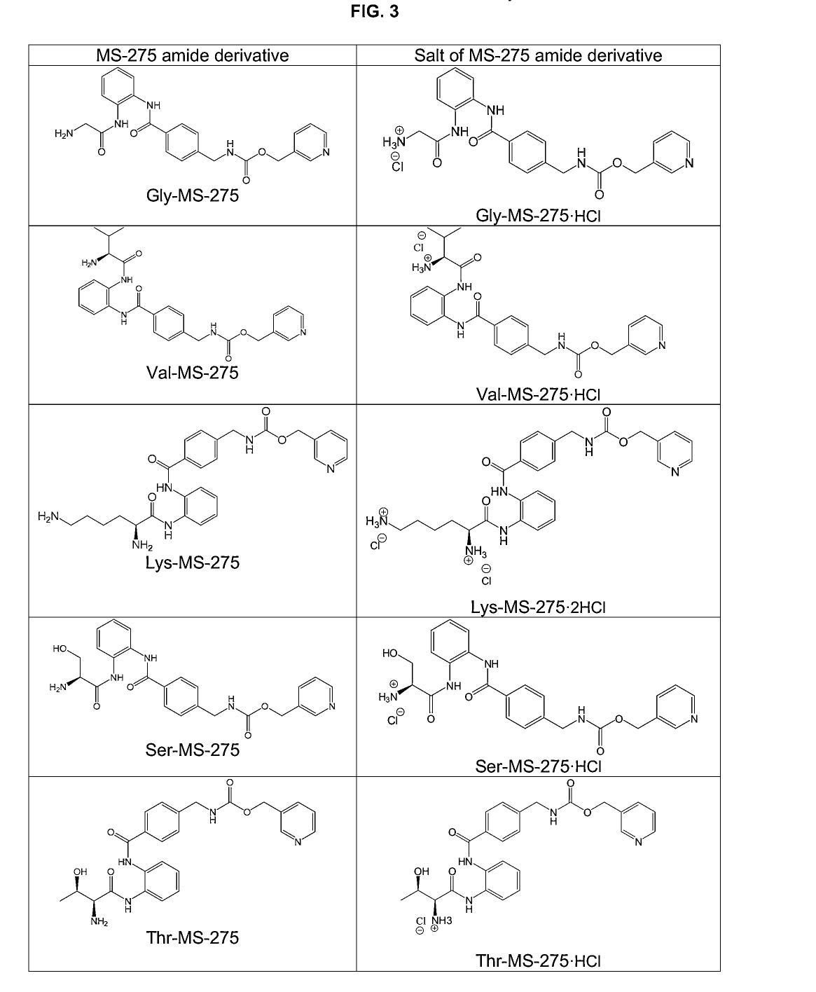

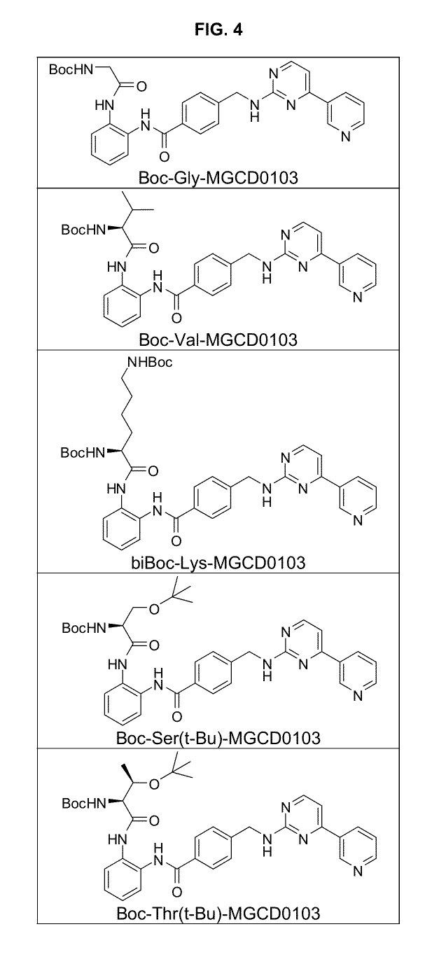

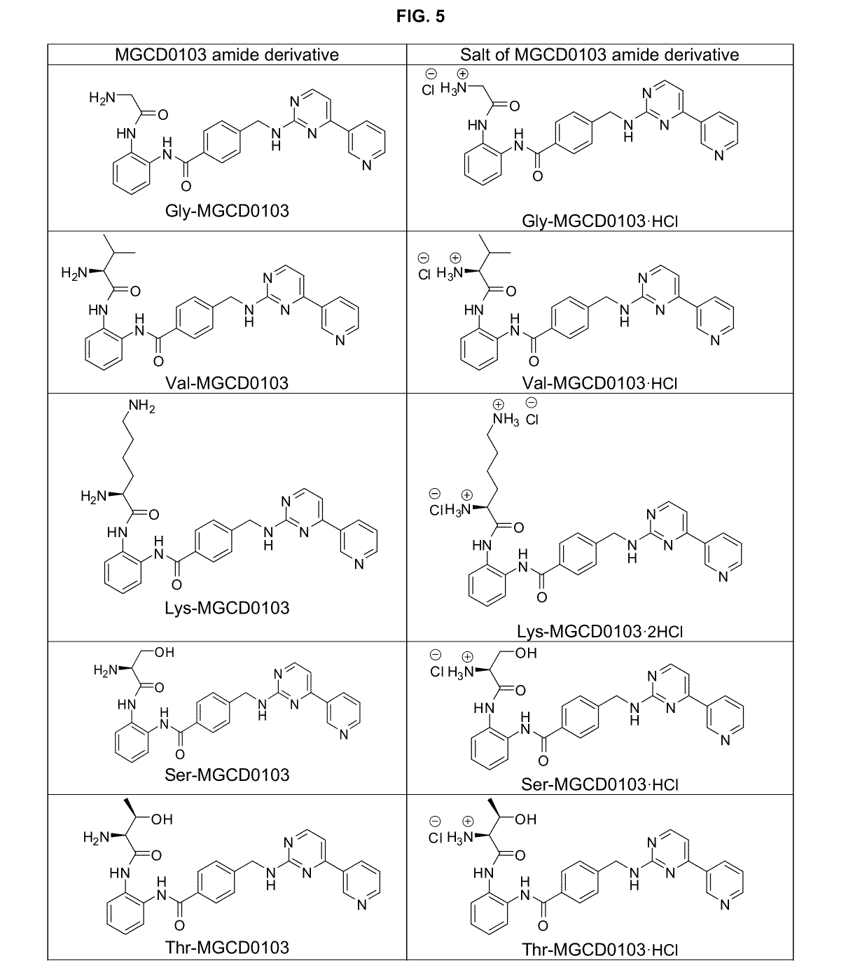

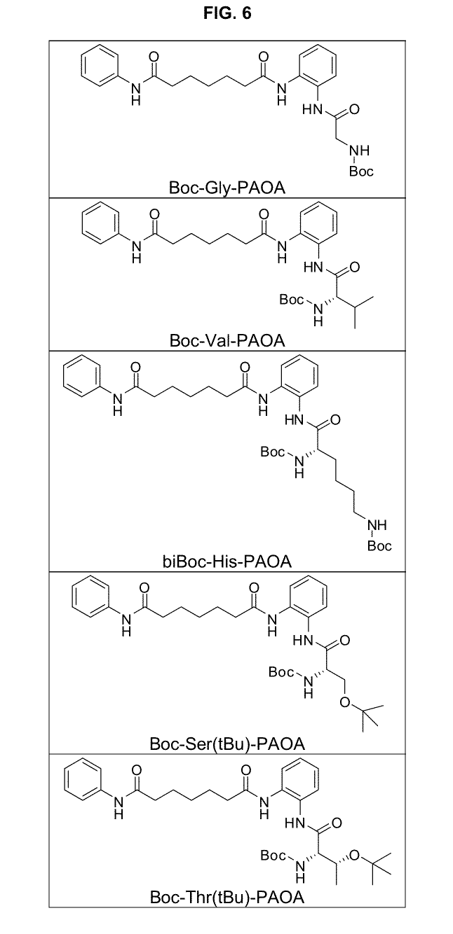

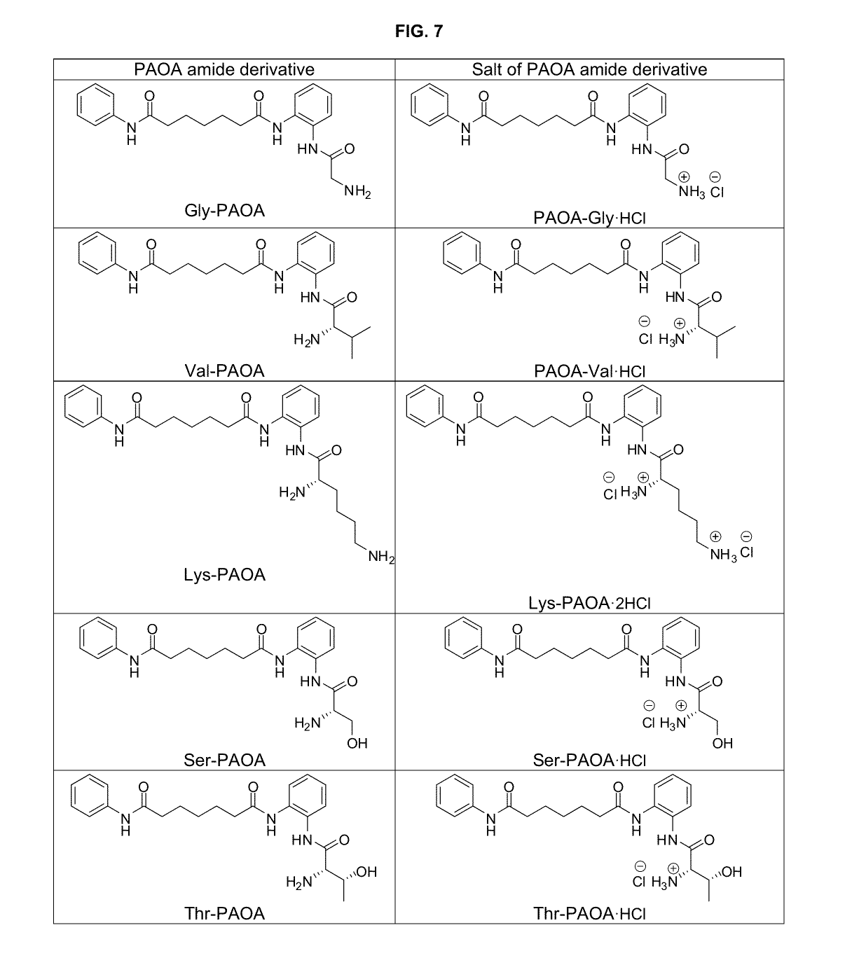

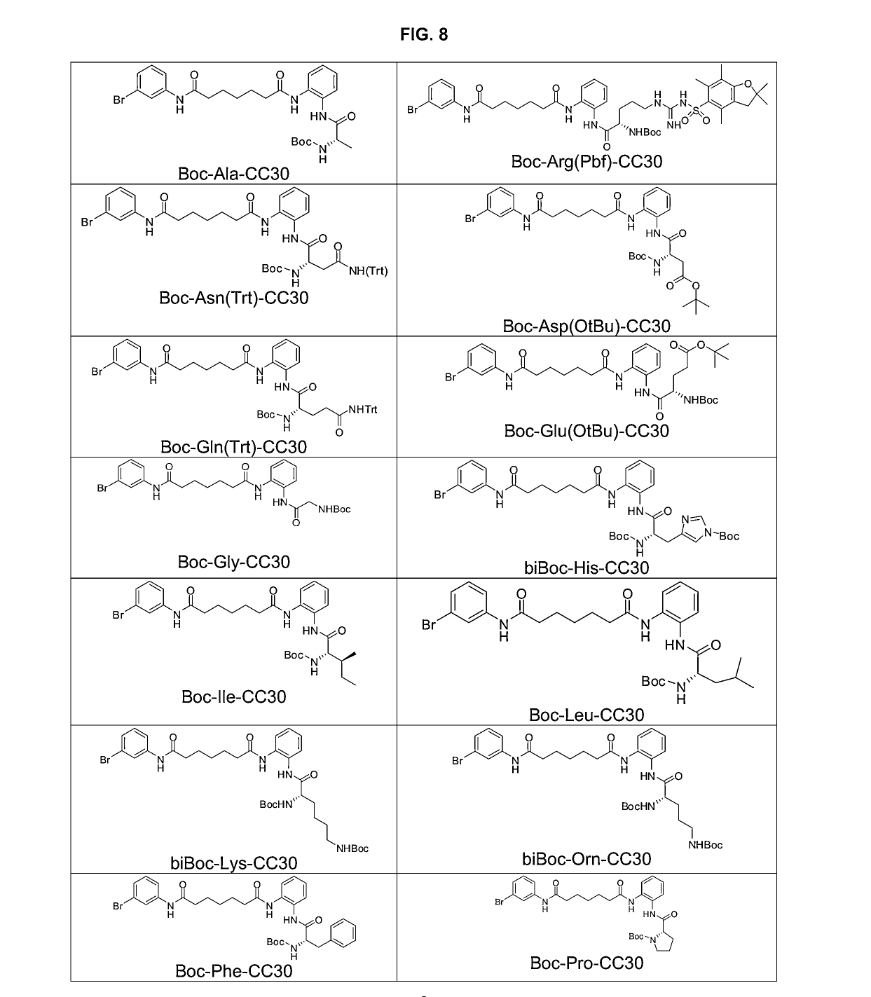

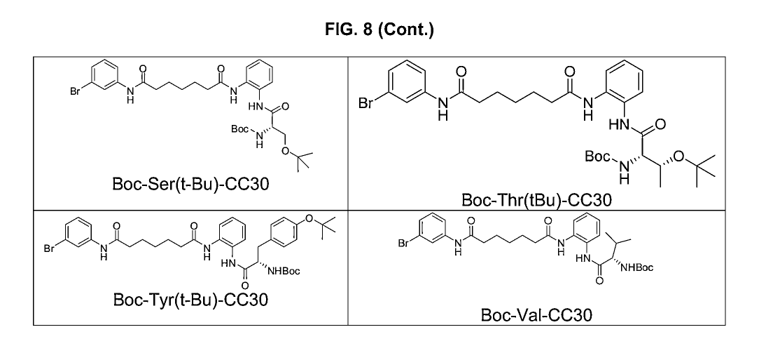

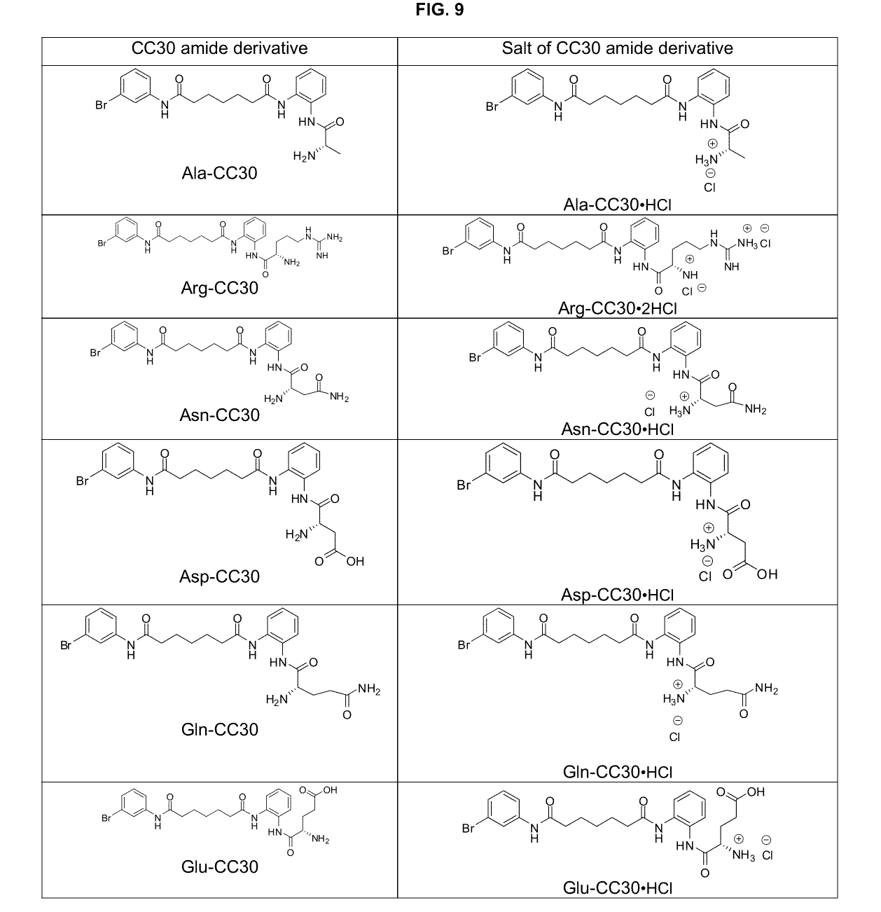

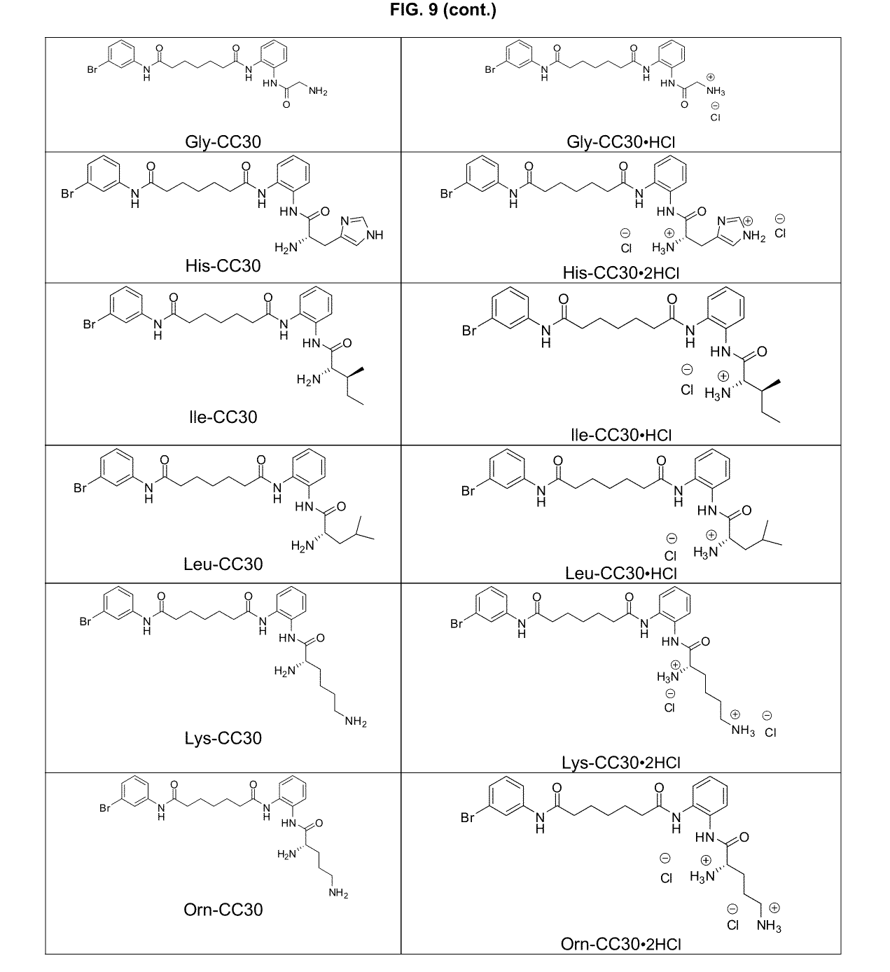

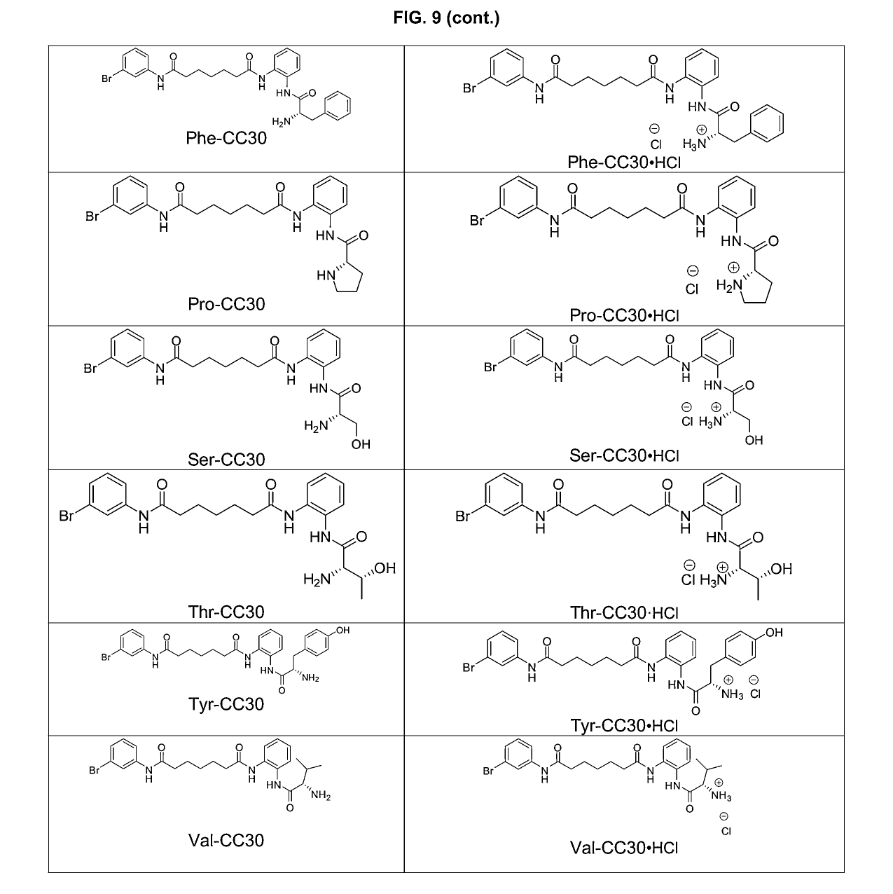

Another aspect of the invention relates to a compound comprising a structure of Structure X, including crystals, stereoisomers, pharmaceutically acceptable solvates, and pharmaceutically acceptable salts thereof, further including mixtures thereof in all ratios, selected from the group comprising of Val-MS-275, Lys-MS-275, Ser-MS-275, Thr-MS-275, Gly-MGCD0103, Val-MGCD0103, Lys-MGCD0103, Ser-MGCD0103, Thr-MGCD0103, Gly-PAOA, Val-PAOA, Lys-PAOA, Ser-PAOA, Thr-PAOA, Ala-CC30, Arg-CC30, Asn-CC30, Asp-CC30, Gln-CC30, Glu-CC30, Gly-CC30, His-CC30, Ile-CC30, Leu-CC30, Lys-CC30, Orn-CC30, Phe-CC30, Pro-CC30, Ser-CC30, Thr-CC30, Tyr-CC30, Val-CC30, and CC30-Suc-OH (FIGS. 3, 5, 7 and 9).

Another aspect of the invention relates to a compound comprising a structure of Structure X, including crystals, stereoisomers, pharmaceutically acceptable solvates, and pharmaceutically acceptable salts thereof, further including mixtures thereof in all ratios, wherein:

Structure PR is Structure II:

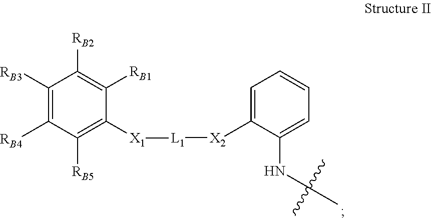

and

R_{B1}˜R_{B5} are independently selected from the group consisting of hydrogen, halogen (e.g. F, Cl, Br, and/or I) and haloalkyl (e.g. trifluoromethyl); and

X₁, X₂, L₁, X, m, Rx and Ry are defined the same as above.

In one embodiment, R_{B1}-R_{B5} are hydrogen; in a further embodiment, —X₁-L₁-X₂— is —C(═O)—NH-L₁-C(═O)NH—.

In one embodiment, R_{B1}-R_{B5} are independently selected from the group consisting of hydrogen and bromine, wherein at least one of R_{B1}-R_{B5} is bromine.

In another embodiment, R_{B1}-R_{B5} are independently selected from the group consisting of hydrogen and fluorine, wherein at least one of R_{B1}-R_{B5} is fluorine.

In another embodiment, R_{B1}-R_{B5} are independently selected from the group consisting of hydrogen and chlorine, wherein at least one of R_{B1}-R_{B5} is chlorine.

In another embodiment, R_{B3} and/or R_{B4} are/is haloalkyl (e.g. trifluoromethyl) or halogen (e.g. F, Cl, Br, and/or I); in a further embodiment, R_{B1}, R_{B2}, R_{B5}, and R_{B6} are hydrogen.

In another embodiment, R_{B3} is a bromine; in a further embodiment, R_{B1}, R_{B2}, R_{B4}, and R_{B5} are hydrogen.

In another embodiment, R_{B3} is a fluorine; in a further embodiment, R_{B1}, R_{B2}, R_{B4}, and R_{B5} are hydrogen.

In another embodiment, R_{B3} is a chlorine; in a further embodiment, R_{B1}, R_{B2}, R_{B4}, and R_{B5} are hydrogen.

In another embodiment, R_{B4} is a bromine; in a further embodiment, R_{B1}, R_{B2}, R_{B3}, and R_{B5} are hydrogen.

In another embodiment, R_{B4} is a fluorine; in a further embodiment, R_{B1}, R_{B2}, R_{B3}, and R_{B5} are hydrogen.

In another embodiment, R_{B4} is a chlorine; in a further embodiment, R_{B1}, R_{B2}, R_{B3}, and R_{B5} are hydrogen.

In another embodiment, L₁ is —(CH₂)ₙ—, wherein n is 4, 5, 6, 7, or 8.

In another embodiment, —X₁-L₁-X₂— is —NHC(═O)-L₁-C(═O)NH—.

In another embodiment, —X₁-L₁-X₂— is —C(═O)—NH-L₁-C(═O)NH—.

In another embodiment, X is C, and m is 1, Rx is nothing; and Ry is an alkyl or alkyl carboxyl.

In another embodiment, X is C, and m is 1, Rx is nothing; Ry is

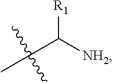

and R₁ is selected from the group consisting of H, alkyl, and alkyl further substituted with a substituent selected from the group consisting of aryl, heteroaryl, amino, hydroxide, hydroxide aryl, hydroxide carbonyl, amino carbonyl, thiol, alkyl-S—, and guanidinyl.

Another aspect of the invention relates to a compound comprising a structure of Structure X, including crystals, stereoisomers, pharmaceutically acceptable solvates, and pharmaceutically acceptable salts thereof, further including mixtures thereof in all ratios, wherein:

Structure PR is Formula VI:

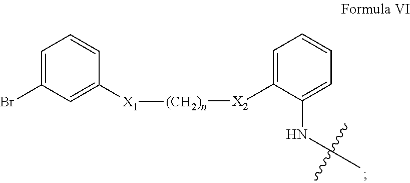

and

n, X₁, X₂, X, m, Rx and Ry are defined the same as supra.

In certain embodiments, n is 4, 5, 6, 7, or 8.

In certain embodiments, —X₁—(CH₂)ₙ—X₂— is —NHC(═O)—(CH₂)ₙ—C(═O)NH—.

In certain embodiments, —X₁—(CH₂)ₙ—X₂— is —C(═O)—NH— (CH₂)ₙ—C(═O)NH—.

In another embodiment, X is C, and m is 1, Rx is nothing; and Ry is an alkyl or alkyl carboxyl.

In another embodiment, X is C, and m is 1, Rx is nothing; Ry is

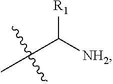

and R₁ is selected from the group consisting of H, alkyl, and alkyl further substituted with a substituent selected from the group consisting of aryl, heteroaryl, amino, hydroxide, hydroxide aryl, hydroxide carbonyl, amino carbonyl, thiol, alkyl-S—, and guanidinyl.

Another aspect of the invention relates to a compound comprising a structure of Structure X, including crystals, stereoisomers, pharmaceutically acceptable solvates, and pharmaceutically acceptable salts thereof, further including mixtures thereof in all ratios, wherein:

Structure PR is Structure VIII:

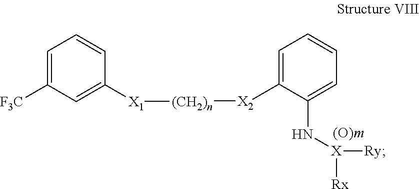

and

n, X₁, X₂, X, m, Rx and Ry are defined the same as above.

In certain embodiments, n is 4, 5, 6, 7, or 8.

In certain embodiments, —X₁—(CH₂)ₙ—X₂— is —NHC(═O)—(CH₂)ₙ—C(═O)NH—.

In certain embodiments, —X₁—(CH₂)ₙ—X₂— is —C(═O)—NH—(CH₂)ₙ—C(═O)NH—.

In another embodiment, X is C; m is 1; Rx is nothing; and Ry is an alkyl or alkyl carboxyl.

In another embodiment, X is C; m is 1; Rx is nothing; Ry is

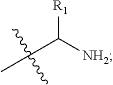

and R₁ is selected from the group consisting of H, alkyl, and alkyl further substituted with a substituent selected from the group consisting of aryl, heteroaryl, amino, hydroxide, hydroxide aryl, hydroxide carbonyl, amino carbonyl, thiol, alkyl-S—, and guanidinyl.

As used herein, an “alkyl” group is a functional group derived from a straight or branched chain hydrocarbon by removing one or two hydrogens from any one or more carbon atoms. An alkyl group also may have one or more unsaturated carbon-carbon bond (e.g. —C═C—, —C≡C—) in the chain structure.

As used herein an “aryl” or an “aryl group” is a functional group derived from an aromatic hydrocarbon by removing one or more hydrogen atoms from any one or two carbon ring atoms, wherein the aromatic hydrocarbon optionally includes an alkyl linker through which it may be attached, preferably a C₁-C₆ alkyl linker as defined above. Such a ring may be optionally fused to one or more other aryl ring(s). Examples of aromatic hydrocarbons include, without limitation, benzene, naphthalene, biphenyl, imidazole, and anthracene. More specifically, when an aromatic hydrocarbon is benzene, the corresponding aryl group can be phenyl or a structure selected from the group consisting of Structure A1, Structure A2, and Structure A3:

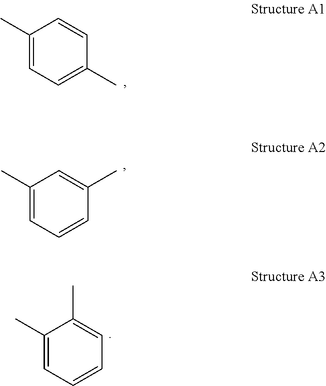

As used herein a “heteroaryl” or a “heteroaryl group” is a functional group derived from a heteroaromatic compound by removing one or more hydrogen atoms from one or two carbon ring atoms at any position of the ring, wherein the heteraromatic hydrocarbon optionally includes an alkyl linker through which it may be attached, preferably a C₁-C₆ alkyl linker as defined above. Such a ring may be optionally fused to one or more other aryl and/or heteroaryl ring(s). Examples of heteroaromatic compounds include, without limitation, pyridine, and pyrimidylene. More specifically, when a heteroaromatic compound is pyridine, the corresponding heteroaryl group can be pyridyl, or have a structure selected from the group consisting of Structure B1, Structure B2, Structure B3, Structure B4, Structure B5, Structure B6, and Structure B7:

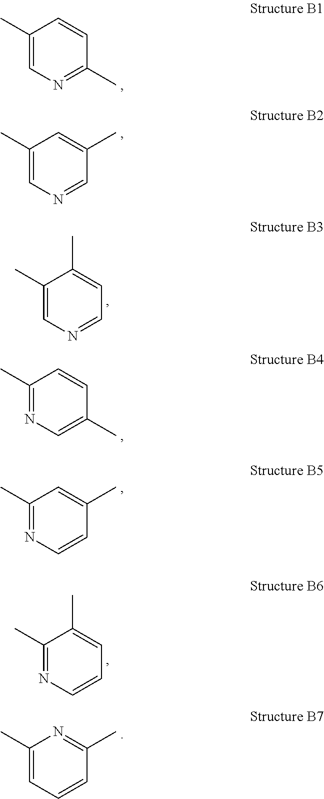

As used herein, a substituted functional group is the functional group further substituted with one or more substitutions at any one or more positions. Examples of substitutions include, without limitation, F, Cl, Br, I, alkyl, haloalkyl (e.g. trifluoromethyl), hydroxyl, amino, alkoxy, alkylamino, alkylcarbonylamino, aryl, heteroaryl, cycloalkyl, and heterocycloalkyl. More specifically, when the functional group has a ring, the one or more substitution may be at any one or more ring atoms as well.

As used herein, the term “halogen” or “halo” refers to fluorine (F), chlorine (CI), bromine (Br) or iodine (I).

As used herein, the term “haloalkyl” refers to an alkyl group wherein one or more hydrogen and/or carbon atoms are substituted with halogen atom.

As used herein, a compound or a composition that is “pharmaceutically acceptable” is suitable for use in contact with the tissue or organ of a biological subject without excessive toxicity, irritation, allergic response, immunogenicity, or other problems or complications, commensurate with a reasonable benefit/risk ratio. If said compound or composition is to be used with other ingredients, said compound or composition is also compatible with said other ingredients.

As used herein, the term “solvate” refers to a complex of variable stoichiometry formed by a solute (e.g., compounds disclosed herein) and a solvent. Such solvents for the purpose of the invention may not interfere with the biological activity of the solute. Examples of suitable solvents include, but are not limited to, water, aqueous solution (e.g. buffer), methanol, ethanol and acetic acid. Preferably, the solvent used is a pharmaceutically acceptable solvent. Examples of suitable pharmaceutically acceptable solvents include, without limitation, water, aqueous solution (e.g. buffer), ethanol and acetic acid. Most preferably, the solvent used is water or aqueous solution (e.g. buffer). Examples for suitable solvates are the mono- or dihydrates or alcoholates of the compound according to the invention.

As used herein, pharmaceutically acceptable salts of a compound refers to any pharmaceutically acceptable acid and/or base additive salt of the compound (e.g. CC30 amide derivatives). Suitable acids include organic and inorganic acids. Suitable bases include organic and inorganic bases. Examples of suitable inorganic acids include, but are not limited to: hydrochloric acid, hydrofluoric acid, hydrobromic acid, hydroiodic acid, sulfuric acid and boric acid. Examples of suitable organic acids include but are not limited to: acetic acid, trifluoroacetic acid, formic acid, oxalic acid, malonic acid, succinic acid, tartaric acid, maleic acid, fumaric acid, methanesulfonic acid, trifluoromethanesulfonic acid, benzoic acid, glycolic acid, lactic acid, citric acid and mandelic acid. Examples of suitable inorganic bases include, but are not limited to: hydroxides of metal (e.g. alkali metals, alkaline earth metals, etc.), oxides of metal (e.g. alkali metals, alkaline earth metals, etc.), ammonia, and hydrazine. Examples of suitable organic bases include, but are not limited to, methylamine, ethylamine, trimethylamine, triethylamine, ethylenediamine, hydroxyethylamine, morpholine, piperazine and guanidine. The invention further provides for the hydrates and polymorphs of all of the compounds described herein.

The compounds disclosed herein may contain one or more chiral atoms, or may otherwise be capable of existing as two or more stereoisomers, which are usually enantiomers and/or diastereomers. Unless otherwise specified, an amino acid referred herein has a L-configuration. Accordingly, the compounds disclosed herein include mixtures of stereoisomers or mixtures of enantiomers, as well as purified stereoisomers, purified enantiomers, stereoisomerically enriched mixtures, or enantiomerically enriched mixtures. The compounds disclosed herein also include the individual stereoisomers of the compound represented by the structure of the CC30 amide derivatives above as well as any wholly or partially equilibrated mixtures thereof. The compounds disclosed herein also cover the individual stereoisomers of the compound represented by the structure of CC30 amide derivatives above as mixtures with stereoisomers thereof in which one or more chiral centers are inverted. Also, it is understood that all tautomers and mixtures of tautomers of the structure of CC30 amide derivatives are included within the scope of the structure of CC30 amide derivatives and preferably the structures corresponding thereto.

Racemates obtained can be resolved into the stereoisomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as camphorsulfonic acid. Also advantageous is enantiomer resolution with the aid of a column filled with an optically active resolving agent. The diastereomer resolution can also be carried out by standard purification processes, such as, for example, chromatography or fractional crystallization.

It is also possible to obtain optically active compounds comprising the structure of the compounds disclosed herein by the methods described above by using starting materials which are already optically active.

Preparation of Amide Derivatives Disclosed Herein.

An amide derivative of an aniline-related compound may be prepared by conventional organic synthesis. For example, the amide derivative can be prepared by reacting the aniline-related compound with a suitable acid, wherein the other reactive groups are protected (e.g. amino group protected by butoxycarbonyl (Boc), triphenylmethyl (Trt), or 2,2,4,6,7-pentamethyl-2,3-dihydrobenzofuran-5-ylsulfonyl (Pbf); hydroxyl group protected by t-butyl (t-Bu or tBu); and carboxyl group protected by t-butyloxy (OtBu)) to avoid undesired reactions. In certain embodiment, the synthesis is carried out in the presence of coupling agent e.g. HBTU (O-(benzotriazol-1-yl)-N,N,N′,N′-tetramethyluronium hexafluorophosphate) and a base (e.g. DIEA (N,N-diisopropylethylamine)).

The amide derivatives prepared, if having protecting groups (“amide derivative (protected)”), may be further converted to the unprotected amide derivatives. The amide derivatives prepared can also be further converted to pharmaceutically acceptable solvates thereof, pharmaceutically acceptable salts thereof, or any mixtures thereof. The amide derivatives prepared can also be further crystallized to provide crystals thereof; or further separated to provide stereoisomers thereof with more than 50% purity of a specific stereoisomer, more than 70% purity of a specific stereoisomer, or more than 90% purity of a specific stereoisomer. The amide derivatives may also be mixed to provide any mixtures thereof at any rations as desired.

Solubility of Amide Derivatives Disclosed Herein.

In certain embodiments, the water solubility of amide derivatives of an aniline-related compound is higher than that of the corresponding aniline-related compound. An acid salt of the amide derivatives (e.g. HCl salt) may also provide enhanced water solubility of the amide derivatives. A base metal salt of the amide derivatives (e.g. alkali metal such as Li, Na, and K) may further enhance the water solubility of the amide derivatives.

Unexpectedly, in certain embodiments, amides with hydrophobic side chains (e.g. Leu-, Val- etc.) may provide similar or even better enhancement in water solubility compared with glycine amide derivatives of the same parent compound.

II. Pharmaceutical Compositions

As used herein, a pharmaceutical composition comprises a therapeutically effective amount of one or more compounds disclosed herein (e.g. CC30 amide derivatives). In certain embodiments, the pharmaceutical composition further comprises a pharmaceutically acceptable carrier.

As used herein, a “therapeutically effective amount,” “therapeutically effective concentration” or “therapeutically effective dose” is an amount which, as compared to a corresponding subject who has not received such amount, results in improved treatment, healing, prevention, or amelioration of a disease, disorder, or side effect, or a decrease in the rate of advancement of a disease or disorder.

A “pharmaceutically acceptable carrier” is a pharmaceutically-acceptable material, composition or vehicle, such as a liquid or solid filler, diluent, excipient, solvent or encapsulating material, involved in carrying or transporting an active ingredient from one location, body fluid, tissue, organ (interior or exterior), or portion of the body, to another location, body fluid, tissue, organ, or portion of the body. Each carrier is “pharmaceutically acceptable” in the sense of being compatible with the other ingredients, e.g., the compounds described herein or other ingredients, of the formulation and suitable for use in contact with the tissue or organ of a biological subject without excessive toxicity, irritation, allergic response, immunogenicity, or other problems or complications, commensurate with a reasonable benefit/risk ratio.

In one embodiment, the pharmaceutical composition administered is made into kits for producing a single-dose administration unit. The kits may each contain both a first container having dried components and a second container having a formulation comprising a pharmaceutically acceptable carrier (e.g. an aqueous formulation). Also included within the scope of this invention are kits containing single and multi-chambered pre-filled syringes (e.g., liquid syringes and lyosyringes).

III. Methods of Using the Compounds and/or Compositions Disclosed Herein

Another aspect of the invention relates a method for treating a condition of a subject comprising administering a therapeutically effective amount of at least one compound and/or one composition disclosed herein to the subject, wherein the condition is a condition treatable by the corresponding parent compound of the compound disclosed herein, e.g., for the treatment of cancer or a condition regulatable by a transcription factor and/or cofactor.

Another aspect of the present disclosure relates to the use of one or more compounds disclosed herein or compositions or pharmaceutical formulations thereof in the manufacture of a medicament for the treatment of cancer or a condition regulatable by a transcription factor and/or cofactor. For this aspect, the compounds, compositions, and formulations are the same as disclosed above, and the treatment of cancer is the same as described supra.

For example, a compound having Structure X as disclosed herein can be used to treat a condition treatable in a subject by the corresponding parent compound having Structure PH:

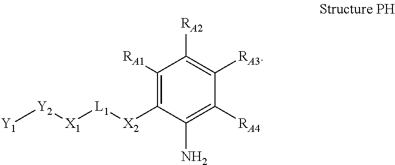

Thus, if the parent compound can be used to treat a condition regulatable by a transcription factor and/or cofactor, the amide derivative thereof can also be applied to the similar use.

Optimal dosages to be administered may be determined by those skilled in the art, such as those disclosed in the Physician's Desk Reference, 41st Ed., Publisher Edward R. Barnhart, N.J. (1987), which is herein incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### Preparation of the Amide Derivatives of Aniline-Related Compounds Disclosed Herein

Several amide derivatives of various aniline-related compound were prepared by reacting the corresponding aniline-related compound and acid (with other reactive groups protected (e.g. amino group protected by Boc, Trt or Pbf; hydroxyl group protected by t-Bu; and carboxyl group protected by OtBu)) in DMF, in the presence of HBTU and DIEA.

I. Preparation of MS-275 Amide Derivatives.

To a solution of the acid in DMF (20 mL) were added HBTU, and DIEA. The reaction mixture was stirred at 10° C. for 10 min. MS-275 was added to the reaction solution. The reaction mixture was stirred for 12 h at room temperature. The reaction was quenched with 50 mL water. The solution was extracted with ethyl acetate (EA, 100 mL×2), the organic layers were combined, dried with anhydrous sodium sulfate and concentrated under reduced pressure. The residue was purified by silica gel chromatography column with ethyl acetate to obtain the amide product. The reactions were summarized in Table 2.

TABLE 2
Preparation of MS-275 amide derivatives (protected)

| MS-275 Amide | | | | | | |
|---|---|---|---|---|---|---|
| Derivative | MS-275 | | Acid | HBTU | DIEA | Yield |
| (protected) | (mmol) | Acid | (mmol) | (mmol) | (mmol) | (%) |
| Boc-Gly- | 0.266 | Boc-Gly-OH | 0.266 | 0.266 | 0.53 | 98 |
| MS-275 | | | | | | |
| Boc-Val- | 0.46 | Boc-Val-OH | 0.55 | 0.55 | 1.66 | 51.7 |
| MS-275 | | | | | | |
| biBoc-Lys- | 0.27 | biBoc-Lys-OH | 0.32 | 0.32 | 0.96 | 52.6 |
| MS-275 | | | | | | |
| Boc-Ser(t-Bu)- | 0.35 | Boc-Ser(t-Bu)—OH | 0.42 | 0.42 | 1.26 | 69.2 |
| MS-275 | | | | | | |
| Boc-Thr(t-Bu)- | 0.30 | Boc-Thr(t-Bu)—OH | 0.36 | 0.36 | 1.2 | 54.3 |
| MS-275 | | | | | | |

The protection groups were removed by acid, e.g. via treatment of HCl (g) in THF at 0° C. Similar reaction also afford a hydrochloride salt of the amide derivative.

To a solution of MS275 amide derivative (protected) in 30 mL of THF was passed into HCl (g) at 0° C. The reaction was stirred for 30 min at 0° C. and filtered to obtain a solid. The solid was washed with ethyl acetate to obtain the hydrochloride of the amide derivative. Table 3 summaries the preparation of the hydrochloride salt of the amide derivatives (and deprotection reaction when applicable).

TABLE 3
Preparation and characterization of hydrochloride salts of certain amide derivatives

| | | | Yield of the MS-275 | |
|---|---|---|---|---|
| | | | Amide Derivative | |
| MS-275 Amide | | MS-275 Amide | (hydrochloride salt) | LC-MS of the |
| Derivative | MS-275 Amide | Derivative | based on the conversion | MS-275 Amide |
| (hydrochloride | Derivative | (protected) | of MS-275 Amide | Derivative |
| salt) | (protected) | mmol | Derivative (protected) (%) | [M + H]+ |
| Gly-MS-275•HCl | Boc-Gly-MS-275 | 0.266 | 81.2 | 434.1852 |
| Val-MS-275•HCl | Boc-Val-MS-275 | 0.238 | 90.2 | 476.2260 |
| Lys-MS-275•2HCl | biBoc-Lys-MS-275 | 0.14 | 86.7 | 505.2521 |
| Ser-MS-275•HCl | Boc-Ser(t-Bu)-MS-275 | 0.29 | 63.2 | 464.1929 |
| Thr-MS-275•HCl | Boc-Thr(t-Bu)-MS-275 | 0.19 | 92.2 | 478.2060 |

The ¹H-NMR (500 MHz, d6-DMSO) of the MS-275 amide derivative (hydrochloride salt) (δ) were:

Gly-MS-275.HCl: 3.82 (d, J=5.5 Hz, 2H), 4.30 (d, J=6.0 Hz, 2H), 5.02 (s, 2H), 7.23-7.26 (m, 2H), 7.39-7.41 (d, J=8.0 Hz, 2H), 7.64-7.67 (m, 2H), 7.79-7.82 (m, 1H), 8.01-8.08 (m, 3H), 8.22 (m, 4H), 8.74-8.79 (m, 2H), 9.90 (s, 1H), 10.33 (s, 1H);

Val-MS-275.HCl: 0.95 (d, J=7.0 Hz, 6H), 2.15-2.19 (m, 1H), 3.90-3.92 (m, 1H), 4.30 (d, J=6.0 Hz, 2H), 5.21 (s, 2H), 7.25-7.27 (m, 2H), 7.39 (d, J=8.0 Hz, 2H), 7.57-7.59 (m, 1H), 7.59 (s, 1H), 7.86 (s, 1H), 8.07-8.08 (m, 3H), 8.29-8.35 (m, 4H), 8.77-8.81 (m, 2H), 10.02 (s, 1H), 10.65 (s, 1H);

Lys-MS-275.2HCl: 1.36-1.41 (m, 2H), 1.80-1.83 (m, 2H), 2.61-2.64 (m, 2H), 3.40-3.43 (m, 1H), 3.59-3.61 (m, 2H), 4.31-4.32 (d, J=6.0 Hz, 2H), 5.18 (s, 2H), 7.26-7.28 (m, 2H), 7.40 (d, J=8.0 Hz, 2H), 7.60-7.65 (m, 2H), 7.70-7.72 (m, 1H), 7.86 (s, 3H), 8.05-8.06 (m, 3H), 8.11-8.13 (m, 1H), 8.38 (s, 3H), 8.69 (d, J=4.5 Hz, 1H), 8.74 (s, 1H), 9.97 (s, 1H), 10.64 (s, 1H);

Ser-MS-275.HCl: 3.86 (d, J=4.5 Hz, 2H), 4.13 (s, 1H), 4.31 (d, J=6.0 Hz, 2H), 5.22 (s, 2H), 7.26-7.28 (m, 2H), 7.40-7.42 (m, 2H), 7.62-7.64 (m, 1H), 7.67-7.69 (m, 1H), 7.83 (s, 1H), 8.03-8.07 (m, 2H), 8.09 (s, 1H), 8.25 (s, 1H), 8.27-8.31 (m, 3H), 8.76-8.81 (m, 2H), 9.90 (s, 1H), 10.42 (s, 1H); and

Thr-MS-275.HCl: 1.17 (d, J=6.5 Hz, 3H), 3.90-3.91 (m, 2H), 4.02-4.05 (m, 2H), 4.31 (d, J=6.0 Hz, 2H), 5.177 (s, 2H), 7.27-7.29 (m, 2H), 7.39-7.41 (m, 2H), 7.63-7.66 (m, 3H), 8.00-8.03 (m, 3H), 8.27 (s, 2H), 8.67-8.74 (m, 2H), 9.87 (s, 1H), 10.32 (s, 1H).

II. Preparation of MGCD0103 Amide Derivatives.

To a solution of the acid in DMF (10 mL) were added HBTU, and DIEA. After 30 min, MGCD0103 was added to the reaction solution. The reaction mixture was stirred overnight at room temperature, then poured into 50 mL water and filtered. The solid obtained was purified by flash C18 chromatography using proper ACN in water for 15 min to obtain the amide product. The reactions were summarized in Table 4.

TABLE 4
Preparation of MGCD0103 amide derivatives (protected)

| MGCD0103 Amide | | | | | | Eluent for flash | |
|---|---|---|---|---|---|---|---|
| Derivative | MGCD0103 | | Acid | HBTU | DIEA | chromatography | Yield |
| (protected) | (mmol) | Acid | (mmol) | (mmol) | (mmol) | (% of ACN in water) | (%) |
| Boc-Gly- | 0.75 | Boc-Gly-OH | 0.83 | 1.13 | 1.5 | 15~40 | 52.5 |
| MGCD0103 | | | | | | | |
| Boc-Val- | 0.5 | Boc-Val-OH | 0.54 | 0.8 | 1.0 | 20~40 | 90.0 |
| MGCD0103 | | | | | | | |
| biBoc-Lys- | 0.65 | biBoc-Lys-OH | 0.7 | 1.0 | 1.3 | 20~40 | 60.0 |
| MGCD0103 | | | | | | | |
| Boc-Ser(t-Bu)- | 0.75 | Boc- | 0.83 | 1.1 | 1.5 | 20~45 | 54.5 |
| MGCD0103 | | Ser(t-Bu)—OH | | | | | |
| Boc-Thr(t-Bu)- | 0.5 | Boc- | 0.54 | 0.7 | 1.0 | 25~45 | 54.5 |
| MGCD0103 | | Thr(t-Bu)—OH | | | | | |

The protection groups were removed by acid, e.g. via treatment of HCl (g) in THF at 0° C. Similar reaction also afford a hydrochloride salt of the amide derivative.

A solution of MGCD0103 amide derivative (protected) in 10 mL of THF was saturated with HCl (g) with stirring for 0.5 h at room temperature. The obtained reaction was poured into ethyl acetate (20 mL) and filtered to provide a solid. The solid was washed with ethyl acetate (20 mL×3), and dried to afford the hydrochloride salt of the MGCD0103 amide derivative.

Table 5 summaries the preparation of the hydrochloride salt of the amide derivatives (and deprotection reaction when applicable).

TABLE 5
Preparation and characterization of hydrochloride salts of MGCD0103 amide derivatives

| | | | Yield of the Hydrochloride | |
|---|---|---|---|---|
| MGCD0103 Amide | | MGCD0103 Amide | Salt of MGCD0103 Amide | LC-MS of the |
| Derivative | MGCD0103 Amide | Derivative | Derivative based on | MGCD0103 Amide |
| (Hydrochloride | Derivative | (protected) | MGCD0103 Amide Derivative | Derivative |
| Salt) | (protected) | mmol | (protected) (%) | [M + H]+ |
| Gly-MGCD0103•HCl | Boc-Gly-MGCD0103 | 0.4 | 97.6 | 454.1936 |
| Val-MGCD0103•HCl | Boc-Val-MGCD0103 | 0.5 | 99.4 | 496.2403 |
| Lys-MGCD0103•2HCl | biBoc-Lys-MGCD0103 | 0.38 | 99.9 | 525.2756 |
| Ser-MGCD0103•HCl | Boc-Ser(t-Bu)-MGCD0103 | 0.43 | 99.9 | 484.2104 |
| Thr-MGCD0103•HCl | Boc-Thr(t-Bu)-MGCD0103 | 0.19 | 92.2 | 498.2198 |

¹H-NMR of the hydrochloride salt of MGCD0103 amide derivative (500 MHz, d6-DMSO) (δ) were:

Gly-MGCD0103.HCl: 3.81 (s, 2H), 4.69 (s, 2H), 7.20-7.23 (m, 2H), 7.41 (s, 1H), 7.45-7.51 (m, 2H), 7.60-7.64 (m, 2H), 7.92-7.95 (m, 1H), 8.02-8.05 (m, 2H), 8.19-8.26 (m, 4H), 8.46 (s, 1H), 8.83-8.91 (m, 2H), 9.39 (s, 1H), 9.93 (s, 1H), 10.22 (s, 1H);

Val-MGCD0103.HCl: 0.91 (s, 6H), 2.11-2.19 (m, 1H), 3.89-3.91 (m, 1H), 4.72 (s, 2H), 7.20-7.23 (m, 2H), 7.41-7.59 (m, 4H), 7.62-7.64 (m, 1H), 8.09 (s, 1H), 8.12-8.16 (m, 2H), 8.19-8.26 (m, 4H), 8.46 (s, 1H), 8.83-8.95 (m, 2H), 9.41 (s, 1H), 10.06 (s, 1H), 10.82 (s, 1H);

Lys-MGCD0103.2HCl: 1.41-1.44 (m, 2H), 1.47-1.53 (m, 2H), 1.82-1.86 (m, 2H), 2.61-2.64 (m, 2H), 4.10-4.12 (m, 1H), 4.71 (s, 2H), 7.22-7.24 (m, 2H), 7.42-7.44 (s, 1H), 7.49-7.53 (m, 2H), 7.56-7.59 (m, 1H), 7.61-7.63 (m, 1H), 7.91-8.06 (m, 4H), 8.12-8.15 (m, 2H), 8.20-8.26 (m, 4H), 8.46 (s, 1H), 8.85-8.89 (m, 2H), 9.39 (s, 1H), 10.02 (s, 1H), 10.81 (s, 1H);

Ser-MGCD0103.HCl: 3.83-3.84 (m, 2H), 4.02-4.04 (m, 1H), 4.71 (s, 2H), 7.22-7.24 (m, 2H), 7.41-7.42 (m, 1H), 7.49-7.53 (m, 2H), 7.61-7.63 (m, 1H), 7.64-7.66 (m, 1H), 7.91-7.93 (m, 1H), 8.03-8.04 (m, 2H), 8.12-8.16 (m, 4H), 8.46 (s, 1H), 8.84-8.89 (m, 2H), 9.38 (s, 1H), 9.89 (s, 1H), 10.45 (s, 1H); and

Thr-MGCD0103.HCl: 1.17 (d, J=6.5 Hz, 3H), 3.90-3.91 (m, 2H), 4.02-4.05 (m, 2H), 4.31 (d, J=6.0 Hz, 2H), 5.177 (s, 2H), 7.27-7.29 (m, 2H), 7.39-7.41 (m, 2H), 7.63-7.66 (m, 3H), 8.00-8.03 (m, 3H), 8.27 (s, 2H), 8.67-8.74 (m, 2H), 9.87 (s, 1H), 10.32 (s, 1H).

III. Preparation of PAOA Amide Derivatives.

To a solution of the acid in DMF were added HBTU, and DIEA. After stirring at room temperature for 30 min, PAOA was added to the reaction solution. The reaction mixture was stirred overnight at room temperature, then poured into 20 mL water and extracted with ethyl acetate (100 mL×3). The organic layers were concentrated and the residue was purified by flash column chromatography (petroleum ether:ethyl acetate=5:1-2:1) to provide the amide product. The reactions were summarized in Table 6.

TABLE 6
Preparation of PAOA amide derivatives (protected)

| | | | | | | | Eluent for flash | |
|---|---|---|---|---|---|---|---|---|
| PAOA Amide | | | | | | | chromatography | |
| Derivative | PAOA | | Acid | DMF | HBTU | DIEA | (petroleum | Yield |
| (protected) | (mmol) | Acid | (mmol) | (mL) | (mmol) | (mmol) | ether:ethyl acetate) | (%) |
| Boc-Gly- | 0.61 | Boc-Gly-OH | 0.74 | 5 | 0.92 | 1.23 | 5:1~2:1 | 98.8 |
| PAOA | | | | | | | | |
| Boc-Val- | 3.037 | Boc-Val-OH | 3.688 | 15 | 4.610 | 6.146 | 5:1~2:1 | 54.1 |
| PAOA | | | | | | | | |
| biBoc-Lys- | 0.61 | biBoc-Lys-OH | 0.74 | 5 | 0.92 | 1.23 | 5:1~2:1 | 92.4 |
| PAOA | | | | | | | | |
| Boc- | 0.61 | Boc- | 0.74 | 5 | 0.92 | 1.23 | 5:1~2:1 | 82.0 |
| Ser(t-Bu)-PAOA | | Ser(t-Bu)—OH | | | | | | |
| Boc- | 0.61 | Boc- | 0.74 | 5 | 0.92 | 1.23 | 5:1~2:1 | 66.7 |
| Thr(t-Bu)-PAOA | | Thr(t-Bu)—OH | | | | | | |

The protection groups were removed by acid, e.g. via treatment of HCl (g) in THF at 0° C. Similar reaction also afford a hydrochloride salt of the amide derivative.

A solution of PAOA amide derivative (protected) in 15 mL of THF was saturated with HCl (g) with stirring for 0.5 h at 0° C. The obtained reaction was dropped into tert-butyl methyl ether (100 mL) and filtered to afford the hydrochloride salt of the PAOA amide derivative.

Table 7 summaries the preparation of the hydrochloride salt of the amide derivatives (and deprotection reaction when applicable).

TABLE 7
Preparation and characterization of hydrochloride salts of PAOA amide derivatives

| | | | Yield of the | |
|---|---|---|---|---|
| PAOA Amide | | | Hydrochloride Salt of | LC-MS of the |
| Derivative | PAOA Amide | PAOA Amide | PAOA Amide Derivative | PAOA Amide |
| (Hydrochloride | Derivative | Derivative | based on PAOA Amide | Derivative |
| Salt) | (protected) | (protected) mg | Derivative (protected) (%) | [M + H]+ |
| Gly-PAOA•HCl | Boc-Gly-PAOA | 293 | 44.9 | 383.2082 |
| Val-PAOA•HCl | Boc-Val-PAOA | 76.6 | 79.9 | 425.2536 |
| Lys-PAOA•2HCl | biBoc-Lys-PAOA | 371 | 97.4 | 454.2838 |
| Ser-PAOA•HCl | Boc-Ser(t-Bu)-PAOA | 343 | 89.6 | 413.2154 |
| Thr-PAOA•HCl | Boc-Thr-PAOA | 172 | 102 | 465.1920 |

¹H-NMR of the hydrochloride salt of PAOA amide derivative (500 MHz, d6-DMSO) (δ) were:

Gly-PAOA.HCl: 1.36-1.40 (m, 2H), 1.64-1.65 (m, 4H), 2.33 (t, J=7.5 Hz, 2H), 2.43 (t, J=7.5 Hz, 2H), 3.86 (s, 2H), 7.01 (t, J=7.5 Hz, 1H), 7.12-7.16 (m, 2H), 7.27 (t, J=7.5 Hz, 2H), 7.58-7.68 (m, 4H), 8.26 (s, 3H), 9.63 (s, 1H), 9.95 (s, 1H), 10.24 (s, 1H);

Val-PAOA.HCl: 1.02 (d, J=7.0 Hz, 6H), 1.33-1.40 (m, 2H), 1.61-1.53 (m, 4H), 2.17-2.24 (m, 1H), 2.32 (t, J=7.5 Hz, 2H), 2.42-2.46 (m, 2H), 3.95-3.99 (m, 1H), 4.17-4.22 (m, 1H), 7.01 (t, J=7.5 Hz, 2H), 7.13-7.19 (m, 2H), 7.27 (t, J=7.5 Hz, 2H), 7.55 (dd, J=7.5, 2.0 Hz, 1H), 7.60 (d, J=7.5 Hz, 1H), 7.67 (d, J=7.5 Hz, 1H), 8.39 (s, 3H), 9.79 (s, 1H), 9.92 (s, 1H), 10.52 (s, 1H);

Lys-PAOA.2HCl: 1.34-1.40 (m, 2H), 1.47-1.53 (m, 2H), 1.61-1.68 (m, 6H), 1.86-1.93 (m, 2H), 2.34 (t, J=7.5 Hz, 2H), 2.46 (t, J=7.5 Hz, 2H), 2.76-2.80 (m, 2H), 4.16-4.20 (m, 1H), 7.01 (t, J=7.5 Hz, 2H), 7.12-7.18 (m, 2H), 7.27 (t, J=7.5 Hz, 2H), 7.58-7.63 (m, 3H), 7.68 (d, J=7.5 Hz, 1H), 8.03 (s, 3H), 8.51 (s, 3H), 9.83 (s, 1H), 10.01 (s, 1H), 10.63 (s, 1H);

Ser-PAOA.HCl: 1.34-1.40 (m, 2H), 1.60-1.66 (m, 4H), 2.33 (t, J=7.5 Hz, 2H), 2.42 (t, J=7.5 Hz, 2H), 3.87-3.96 (m, 2H), 4.16-4.18 (m, 1H), 5.64 (s, 1H), 7.01 (t, J=7.5 Hz, 1H), 7.13-7.18 (m, 2H), 7.27 (t, J=7.5 Hz, 2H), 7.55-7.57 (m, 1H), 7.61 (d, J=7.5 Hz, 1H), 7.67-7.72 (m, 1H), 8.34 (s, 3H), 9.58 (s, 1H), 9.95 (s, 1H), 10.35 (s, 1H); and

Thr-PAOA.HCl: 1.24 (d, J=6.5 Hz, 3H), 1.34-1.40 (m, 2H), 1.62-1.65 (m, 4H), 2.33 (t, J=7.5 Hz, 2H), 2.43 (t, J=7.5 Hz, 2H), 3.98 (t, J=5.0 Hz, 1H), 4.06-4.11 (m, 1H), 5.69 (s, 1H), 7.01 (t, J=7.5 Hz, 1H), 7.12-7.19 (m, 2H), 7.27 (t, J=8.0 Hz, 2H), 7.56 (dd, J=7.5, 1.5 Hz, 1H), 7.60 (d, J=7.5 Hz, 2H), 7.67 (d, J=5.0 Hz, 1H), 8.32 (s, 3H), 9.68 (s, 1H), 9.92 (s, 1H), 10.44 (s, 1H).

IV. Preparation of CC30 Amide Derivatives.

To a solution of the acid (29 mmol, unless otherwise specified) in DMF (200 mL, unless otherwise specified) were added HBTU (37 mmol, unless otherwise specified), and DIEA (50 mmol, unless otherwise specified). After stirring at room temperature for 30 min, CC30 (about 25 mmol) was added to the reaction solution. The reaction mixture was stirred overnight at room temperature, then poured into 200 mL water and extracted with ethyl acetate (200 mL×3). The organic layers were concentrated and the residue was purified by flash column chromatography (PE:ethyl acetate=5:1-2:1) to provide the amide product.

A solution of CC30 amide derivative (protected, all that was obtained from the previous reaction) in 100 mL of THF was saturated with HCl (g) with stirring for 0.5 h at 0° C. The obtained reaction was dropped into ethyl acetate (800 mL) and filtered to afford the hydrochloride salt of the CC30 amide derivative.

The reactions were summarized in Table 8.

TABLE 8
Preparation of CC30 amide derivatives (hydrochloride salt)

| | | | | | | | Yield of the Hydro- | |
|---|---|---|---|---|---|---|---|---|
| | | | | | | | chloride Salt of CC30 | LC-MS of |
| CC30 Amide | | | | | | | Amide Derivative based | CC30 Amide |
| Derivative | CC30 | | Acid | DMF | HBTU | DIEA | on CC30 Amide Derivative | Derivative |
| (hydrochloride salt) | (mmol) | Acid | (mmol) | (mL) | (mmol) | (mmol) | (protected) (%) | [M + H]+ |
| Ala- | 37 | Boc-Ala-OH | 45 | 250 | 56 | 75 | 86 | 475.1364 |
| CC30•HCl | | | | | | | | |
| Arg- | 24.7 | Boc-Arg(Pbf)-OH | 28.5 | 75 | 28.4 | 36.8 | 64 | 560.1969 |
| CC30•2HCl | | | | | | | | |
| Asn- | 17.3 | Boc-Asn(Trt)-OH | 20.7 | 70 | 20.7 | 26 | 94 | 518.3406 |
| CC30•HCl | | | | | | | | |
| Asp- | 25 | Boc- | 30 | 200 | 37 | 50 | 96 | 519.1285 |
| CC30•HCl | | Asp(OtBu)—OH | | | | | | |
| Gln- | 19.7 | Boc-Gln(Trt)-OH | 23.7 | 80 | 23.7 | 29.7 | 97 | 532.1608 |
| CC30•HCl | | | | | | | | |
| Glu- | 24.6 | Boc- | 29.7 | 70 | 29.7 | 36.8 | 93 | 533.1413 |
| CC30•HCl | | Glu(OtBu)—OH | | | | | | |
| Gly- | 24.7 | Boc-Gly-OH | 27.6 | 60 | 27.4 | 37.5 | 94 | 461.1180 |
| CC30•HCl | | | | | | | | |
| His- | 17.3 | biBoc-His-OH | 19.1 | 70 | 20.7 | 25.9 | 46 | 541.1599 |
| CC30•2HCl | | | | | | | | |
| Ile- | 25 | Boc-Ile-OH | 30 | 200 | 37 | 50 | 61 | 517.3956 |
| CC30•HCl | | | | | | | | |
| Leu- | 17.3 | Boc-Leu-OH | 20.7 | 70 | 20.7 | 26 | 83 | 517.1841 |
| CC30•HCl | | | | | | | | |
| Lys- | 17.3 | biBoc-Lys-OH | 20.7 | 70 | 20.7 | 25.9 | 79 | 532.2174 |
| CC30•2HCl | | | | | | | | |
| Orn- | 24.7 | biBoc-Orn-OH | 29.7 | 100 | 29.7 | 37.1 | 90 | 518.1896 |
| CC30•2HCl | | | | | | | | |
| Phe- | 17.3 | Boc-Phe-OH | 19.1 | 70 | 20.7 | 25.9 | 82 | 551.1781 |
| CC30•HCl | | | | | | | | |
| Pro- | 17.3 | Boc-Pro-OH | 19.1 | 70 | 20.7 | 25.9 | 96 | 501.1572 |
| CC30•HCl | | | | | | | | |
| Ser- | 25 | Boc- | 29 | 200 | 37 | 50 | 85 | 491.1277 |
| CC30•HCl | | Ser(t-Bu)—OH | | | | | | |
| Thr- | 25 | Boc- | 29 | 200 | 37 | 50 | 96 | 505.1446 |
| CC30•HCl | | Thr(t-Bu)—OH | | | | | | |
| Tyr- | 24.6 | Boc- | 29.7 | 75 | 29.7 | 37.3 | 75 | 567.1607 |
| CC30•HCl | | Tyr(t-Bu)— OH | | | | | | |
| Val- | 24.7 | Boc-Val-OH | 32.3 | 60 | 32.4 | 37.8 | 66 | 503.1892 |
| CC30•HCl | | | | | | | | |

¹H-NMR of the hydrochloride salt of CC30 amide derivative (500 MHz, d6-DMSO) (δ) were:

Ala-CC30.HCl: 1.35-1.39 (m, 2H), 1.50 (d, J=7.0 Hz, 3H), 1.60-1.67 (m, 4H), 2.34 (t, J=7.5 Hz, 2H), 2.41-2.45 (m, 2H), 4.17-4.22 (m, 1H), 7.15-7.26 (m, 4H), 7.51-7.54 (m, 1H), 7.57-7.59 (m, 1H), 7.61-7.63 (m, 1H), 7.99 (t, J=1.8 Hz, 1H), 8.35 (s, 3H), 9.75 (s, 1H), 10.18 (s, 1H), 10.37 (s, 1H).

Arg-CC30.2HCl: 1.34-1.40 (m; 2H), 1.59-1.67 (m; 6H), 1.85-1.90 (m; 2H), 2.35 (t, J=7.3 Hz; 2H), 2.44 (t, J=7.3 Hz; 2H), 3.19 (d, J=6.5 Hz; 2H), 4.19 (br; 1H), 7.10 (br; 5H), 7.14-7.26 (m; 3H), 7.51 (d, J=8.0 Hz; 1H), 7.58 (dd, J=7.5, 2.0 Hz; 1H), 7.65 (dd, J=8.0, 1.5 Hz; 1H), 7.72 (t, J=5.8 Hz; 1H), 7.96 (t, J=6.8 Hz; 1H), 8.42 (br; 3H), 9.34 (s; 1H), 9.67 (s; 1H), 10.14 (s; 1H), 10.43 (s; 1H).

Asn-CC30.HCl: 1.34-1.37 (m, 2H), 1.62-1.64 (m, 4H), 2.35-2.37 (m, 2H), 2.44-2.50 (m, 2H), 2.87-2.88 (m, 2H), 4.29-4.31 (br, 1H), 7.07-7.25 (m, 6H), 7.33-7.55 (m, 2H), 7.74-7.80 (m, 2H), 8.00 (s, 1H), 8.45-8.46 (m, 3H), 9.44 (s, 1H), 10.26-10.28 (m, 2H).

Asp-CC30.HCl: 1.34-1.41 (m, 2H), 1.62-1.67 (m, 4H), 2.34 (t, J=7.5 Hz, 2H), 2.43 (t, J=7.5 Hz, 2H), 2.96 (dd, J=17.5, 5.5 Hz, 1H), 3.04 (dd, J=17.5, 7.0 Hz, 1H), 4.34 (t, J=6.0 Hz, 1H), 7.14-7.26 (m, 4H), 7.51 (t, J=8.5 Hz, 1H), 7.67 (d, J=12.5 Hz, 1H), 7.99 (s, 1H), 8.48 (s, 3H), 9.48 (s, 1H), 10.17 (s, 1H), 10.24 (s, 1H), 12.93 (s, 1H).

Gln-CC30.HCl: 1.34-1.37 (m, 2H), 1.60-1.64 (m, 4H), 2.06-2.08 (m, 2H), 2.29-2.35 (m, 4H), 2.41-2.43 (m, 2H), 4.13-4.14 (s, 1H), 6.94 (s, 1H), 7.14-7.26 (m, 4H), 7.49-7.51 (m, 2H), 7.58-7.63 (m, 2H), 7.98 (s, 1H), 8.40-8.43 (m, 3H), 9.58 (s, 1H), 10.12 (s, 1H), 10.28 (s, 1H).

Glu-CC30.HCl: 1.33-1.39 (m; 2H), 1.60-1.66 (m; 4H), 2.08-2.12 (m; 2H), 2.34 (t, J=7.3 Hz; 2H), 2.41-2.46 (m; 4H), 4.16 (br; 1H), 7.14-7.26 (m; 4H), 7.51 (d, J=8.0 Hz; 1H), 7.54-7.56 (m; 1H), 7.62 (d, J=7.5 Hz; 1H), 7.98 (s; 1H), 8.34 (br; 3H), 9.61 (s; 1H), 10.11 (s; 1H), 10.31 (s; 1H), 12.31 (br, 1H).

Gly-CC30.HCl: 1.37 (quint, J=7.0 Hz; 2H), 1.67 (m; 4H), 2.35 (t, J=7.5 Hz; 2H), 2.42 (t, J=7.0 Hz; 2H), 3.85 (d, J=5.5 Hz; 2H), 7.14-7.17 (m; 2H), 7.19-7.26 (m; 2H), 7.52 (d, J=7.8 Hz; 1H), 7.57-7.59 (m; 1H), 7.66 (br; 1H), 7.98 (s; 1H), 8.22 (br; 3H), 9.57 (br; 1H), 10.14 (br; 1H).

His-CC30.HCl: δ 1.35-1.38 (m, 2H), 1.60-1.63 (m, 4H), 2.30-2.32 (m, 2H), 2.40-2.43 (m, 2H), 3.30-3.34 (dd, J=7.0, 8.5 Hz, 1H), 3.42-3.46 (dd, J=7.0, 8.5 Hz, 1H), 4.56-4.59 (m, 1H), 7.12-7.26 (m, 4H), 7.51-7.53 (m, 2H), 7.57 (s, 1H), 7.66-7.67 (m, 1H), 7.99 (s, 1H), 8.71-8.73 (m, 3H), 9.07 (s, 1H), 9.70 (s, 1H), 10.19 (s, 1H), 10.50 (s, 1H), 14.22 (bs, 1H), 14.45 (bs, 1H).

Ile-CC30.HCl: 0.90 (t, J=7.5 Hz, 3H), 1.00 (d, J=7.0 Hz, 3H), 1.35-1.39 (m, 2H), 1.61-1.65 (m, 4H), 1.95-1.98 (m, 1H), 2.34 (t, J=7.5 Hz, 2H), 2.43-2.47 (m, 2H), 4.02 (t, J=5.0 Hz, 1H), 7.14-7.26 (m, 4H), 7.52-7.55 (m, 2H), 7.68 (d, J=7.5 Hz, 1H), 7.99 (s, 1H), 8.43 (s, 3H), 9.84 (s, 1H), 10.19 (s, 1H), 10.54 (s, 1H).

Leu-CC30.HCl: δ 0.94-0.97 (m, 6H), 1.36-1.37 (m, 2H), 1.61-1.65 (m, 5H), 1.72-1.74 (m, 2H), 2.36-2.38 (t, J=7.5 Hz, 2H), 2.41-2.43 (t, J=7.5 Hz, 2H), 4.09-4.12 (m, 1H), 7.14-7.22 (m, 4H), 7.50-7.54 (t, J=9.5 Hz, 2H), 7.63-7.64 (d, J=7.5 Hz, 1H), 7.98 (s, 1H), 8.42-8.43 (m, 3H), 9.77 (s, 1H), 10.14 (s, 1H), 10.46 (s, 1H).

Lys-CC30.2HCl: δ 1.35-1.37 (m, 2H), 1.42-1.44 (m, 2H), 1.61-1.65 (m, 6H), 1.81-1.84 (m, 2H), 2.34-2.36 (t, J=7.0 Hz, 2H), 2.43-2.46 (t, J=7.0 Hz, 2H), 2.76-2.79 (m, 2H), 4.15-4.17 (m, 1H), 7.12-7.26 (m, 4H), 7.52-7.54 (d, J=8.0 Hz, 1H), 7.58-7.60 (d, J=8.0 Hz, 1H), 7.65-7.66 (d, J=7.5 Hz, 1H), 7.95 (br, 3H), 7.99 (s, 1H), 8.46 (br, 3H), 9.79 (s, 1H), 10.22 (s, 1H), 10.55 (s, 1H).

Orn-CC30.2HCl: 1.36-1.38 (m, 2H), 1.61-1.65 (m, 4H), 1.77-1.79 (m, 2H), 1.82-1.84 (m, 2H), 2.39-2.41 (m, 2H), 2.49-2.50 (m, 2H), 2.93-2.96 (m, 2H), 4.25-4.27 (br, 1H), 7.13-7.26 (m, 4H), 7.52-7.54 (d, J=8.0 Hz, 1H), 7.58-7.60 (d, J=8.0 Hz, 1H), 7.69-7.71 (d, J=7.5 Hz, 1H), 7.99 (s, 1H), 8.04 (br, 3H), 8.54 (br, 3H), 9.77 (s, 1H), 10.23 (s, 1H), 10.64 (s, 1H).

Phe-CC30.HCl: 1.35-1.38 (m, 2H), 1.60-1.63 (m, 4H), 2.30-2.33 (m, 2H), 2.40-2.43 (m, 2H), 3.12-3.14 (d, J=7.5 Hz, 1H), 3.21-3.23 (d, J=7.5 Hz, 1H), 4.36-4.37 (m, 1H), 7.12-7.19 (m, 2H), 7.26-7.28 (m, 2H), 7.31-7.34 (m, 5H), 7.38-7.39 (d, J=8.0 Hz, 1H), 7.49-7.51 (d, J=8.0 Hz, 1H), 7.65-7.66 (d, J=8.0 Hz, 1H), 7.98 (s, 1H), 8.45-8.46 (m, 3H), 9.61 (s, 1H), 10.12 (s, 1H), 10.41 (s, 1H).

Pro-CC30.HCl: 1.33-1.38 (m, 2H), 1.61-1.65 (m, 4H), 1.76 (m, 1H), 1.92-1.93 (m, 2H), 1.95-1.98 (m, 1H), 2.33-2.36 (m, 2H), 2.39-2.42 (m, 2H), 3.25-3.26 (m, 1H), 3.60-3.62 (m, 1H), 4.49-4.51 (m, 1H), 7.14-7.26 (m, 4H), 7.51-7.52 (d, J=8.0 Hz, 1H), 7.51-7.58 (m, 2H), 7.98 (s, 1H), 8.67 (s, 1H), 9.70 (s, 1H), 9.76-9.82 (bs, 1H), 10.17 (s, 1H), 10.29-10.33 (bs, 1H).

Ser-CC30.HCl: 1.33-1.39 (m, 2H), 1.63 (br, 4H), 2.34 (t, J=7.3 Hz, 2H), 2.41 (t, J=7.3 Hz, 2H), 3.86-3.95 (m, 2H), 4.16 (br, 1H), 5.61 (br, 1H), 7.15-7.26 (m, 4H), 7.51 (d, J=8.0 Hz, 1H), 7.54 (d, J=7.5 Hz, 1H), 7.66 (d, J=7.0 Hz, 1H), 7.98 (s, 1H), 8.31 (s, 3H), 9.53-9.55 (m, 1H), 10.16-10.17 (m, 1H), 10.26 (s, 1H).

Thr-CC30.HCl: 1.24 (d, J=6.5 Hz, 3H), 1.33-1.39 (m, 2H), 1.60-1.67 (m, 4H), 2.34 (t, J=7.2 Hz, 2H), 2.42 (t, J=7.2 Hz, 2H), 3.08 (s, 1H), 3.98 (s, 1H), 4.06-4.11 (m, 1H), 5.70 (br, 1H), 7.51 (d, J=8.0 Hz, 1H), 7.54 (dd, J=7.0, 1.5 Hz, 1H), 7.66 (d, J=7.5 Hz, 1H), 7.98 (s, 1H), 8.31 (s, 3H), 9.65 (s, 1H), 10.15 (s, 1H), 10.39 (s, 1H).

Tyr-CC30.HCl: 1.33-1.39 (m; 2H), 1.58-1.65 (m; 4H), 2.08-2.12 (m; 2H), 2.32 (t, J=7.3 Hz; 2H), 2.40-2.43 (m; 2H), 2.99 (dd, J=14.0, 7.0 Hz; 1H), 3.12 (dd, J=14.0, 7.0 Hz; 1H), 4.25 (br; 1H), 6.71 (d, J=7.5 Hz; 2H), 7.11-7.17 (m; 6H), 7.20 (d, J=8.0 Hz; 1H), 7.24 (t, J=8.0 Hz; 1H), 7.41 (dd, J=8.0, 1.5 Hz; 1H), 7.50 (d, J=8.0 Hz; 1H), 7.98 (s; 1H), 8.35 (br; 3H), 9.34 (s; 1H), 9.57 (s; 1H), 10.11 (s; 1H), 10.28 (s; 1H).

Val-CC30.HCl: 1.02-1.04 (m; 6H), 1.36 (quint, J=7.0 Hz; 2H), 1.59-1.66 (m; 4H), 2.17-2.24 (m; 1H), 2.34 (t, J=7.0 Hz; 2H), 2.40-2.47 (m; 2H), 3.94-3.98 (m; 1H), 7.14-7.26 (m; 4H), 7.51 (d, J=8.0 Hz; 1H), 7.55 (dd, J=7.0, 1.5 Hz; 1H); 7.65 (d, J=7.5 Hz; 1H); 7.98 (s; 1H), 8.37 (d, J=3.5 Hz; 3H), 9.75 (s; 1H), 10.14 (s; 1H), 10.44 (s; 1H).

Synthesis of CC30-Glu.Ala

Glu-CC30.HCl (2.516 g, 4.4 mmol) was suspended in water (20 mL), and cooled to 0-5° C. in ice-water bath. To the stirring suspension was dropwise added a solution of NaOH (0.338 g, 8.4 mmol) in water (5 mL). The suspension turned thicker first, and then gradually dissolved to form a clear solution. The resultant solution was filtered through Celite. The filtrate was co-evaporated with MeOH and toluene to dryness. To the residue obtained was added THF (100 mL), and the mixture was stirred at 50° C. for 30 min. After being left at ambient temperature for 16 hours, the solution was filtered through Celite, and the filtrated was concentrated. To the concentrated solution was added methyl t-butyl ether (MTBE) (ca. 100 mL), and provide the product CC30-Glu.Na (2.098 g. Yield: 86%) after removal of the solvent. LC-MS: m/z=533.1451 ([M−Na+2H]⁺).

¹H NMR (500 MHz, DMSO-d₆): δ 1.33-1.39 (m; 2H), 1.58-1.68 (m; 4H), 1.71-1.77 (m; 1H), 1.81-1.88 (m; 1H), 2.04-2.14 (m; 2H), 2.33-2.39 (m; 4H), 3.38 (t, J=6.3 Hz; 1H), 4.96 (br; 2H), 7.05 (td, J=7.8, 1.2 Hz; 1H), 7.12-7.16 (m; 2H), 7.20 (t, J=8.0 Hz; 1H), 7.42 (d, J=7.8 Hz; 1H), 7.68 (d, J=7.8 Hz; 1H), 7.90 (d, J=7.9 Hz; 1H), 8.10 (s; 1H), 9.89 (s; 1H), 11.49 (br; 1H).

Synthesis of CC30-suc-OMe

Monomethyl succinate (HO-Suc-OMe, 3.930 g, 29.7 mmol) and HBTU (11.300 g, 29.8 mmol) were dissolved in DMF (50 mL) at room temperature. To the solution was dropwise added DIPEA (4.805 g, 37.2 mmol) and the solution was stirred at 10-15° C. for 1 h. To the mixture was added CC30 (10.020 g, 24.8 mmol), and the solution was stirred for 16 h at 30° C. TLC showed the reaction was complete. The reaction mixture was poured into water, and extracted twice with EA. The combined organic layers were washed with water, brine, dried over Na₂SO₄, and filtered. The filtrate was concentrated to give an oil. The oil was triturated with EA/MTBE (5:1, 100 mL) to provide a solid. The solid was collected by filtration and dried. The mother liquor was concentrated and triturated with EA/MTBE (10:1, 20 mL) to provide a second batch of solid. The two lots were combined to give the product (CC30-suc-OMe) as a pale yellow solid (11.150 g, 21.5 mmol, Yield: 87%). LC-MS: m/z=518.1306 ([M+H]⁺)

Synthesis of CC30-suc-OH

CC30-suc-OMe (2.457 g, 4.7 mmol) was dissolved in THF (25 mL). To the solution was added a solution of LiOH.H₂O (0.845 g, 20.1 mmol) in water (5 mL). The resultant mixture was brought to 30° C. and stirred for 6 h. THF was removed under reduced pressure and the residue was suspended in water (100 mL) and EA (100 mL). To the mixture was added 3M HCl (ca. 6.5 mL) until all solid was dissolved. The aqueous phase was extracted with EA (50 mL), washed with water, dried over Na₂SO₄, and concentrated to give a residue. The residue was triturated with EA (50 mL) at 45-50° C. for 30 min, and the solid collected by filtration was dried under vacuum at 45° C. to provide the desired product (CC30-suc-OH) (2.067 g, 4.1 mmol. Yield: 86%). LC-MS: m/z=504.1174 ([M+H]⁺).

¹H NMR (500 MHz, DMSO-d₆): δ 1.32-1.38 (m; 2H), 1.58-1.64 (m; 4H), 2.30-2.35 (m; 4H), 2.50-2.58 (m; 4H), 7.08-7.12 (m; 2H), 7.18-7.25 (m; 4H), 7.45-7.47 (m; 2H), 7.55-7.57 (m; 1H), 7.94 (t, J=2.0 Hz; 1H), 9.10 (s; 1H), 9.35 (s; 1H), 10.00 (s; 1H), 12.12 (s; 1H).

Synthesis of CC30-suc-ONa

CC30-suc-OH (0.427 g, 0.85 mmol) was suspended in a THF/water mixture (THF/water=1:1, 10 mL). To the suspension was dropwise added 0.77 M NaOH aqueous solution (ca. 1.1 mL, 0.85 mmol). At the end of addition, most solid was dissolved, and the solution pH was 9-10. The resultant solution was filtered through Celite, concentrated to dryness, and further dried by co-evaporation with MeOH and toluene to provide the sodium salt CC30-suc-ONa (0.468 g, 8.3 mmol; Yield: 98%, corrected with residual toluene in the solid). LC-MS: m/z=504.1179 ([M−Na+2H]⁺).

¹H NMR (500 MHz, DMSO-d₆): δ 1.35-1.41 (m; 2H), 1.62-1.69 (m; 4H), 2.37-2.47 (m; 8H), 7.02-7.08 (m; 1H), 7.15-7.26 (m; 3H), 7.51 (d, J=7.0 Hz; 1H), 7.64 (d, J=8.0 Hz; 1H), 7.84 (d, J=7.5 Hz; 1H), 8.04 (s; 1H), 9.52 (s; 1H), 10.48 (br; 1H), 11.59 (br; 1H).

### Example 2

### Solubility of Various Amide Derivatives

Solubility of a compound was obtained by adding the compound into water of known volume (e.g. 1 mL) until saturated, measuring the amount of the compound added, and obtaining the solubility of the compound in water (mg/mL) (Table 9).

TABLE 9
Solubility of various amide derivatives.

| Structure PR | Amide Derivative | Solubility in Water (mg/mL) |
|---|---|---|
| | CC30 | 0.006 |
| Structure PR-4 | Gly-CC30•HCl | 10 |
| Structure PR-4 | Lys-CC30•2HCl | 750 |
| Structure PR-4 | Val-CC30•HCl | 170 |
| Structure PR-4 | Orn-CC30•HCl | 720 |
| Structure PR-4 | Asn-CC30•HCl | 30 |
| Structure PR-4 | His-CC30•2HCl | 130 |
| Structure PR-4 | Leu-CC30•HCl | 50 |
| Structure PR-4 | Ser-CC30•HCl | 10 |
| Structure PR-4 | Thr-CC30•HCl | 1350 |
| Structure PR-4 | Phe-CC30•HCl | 10 |
| Structure PR-4 | Arg-CC30•HCl | 100 |
| Structure PR-4 | CC30-Glu•Na | >100 |
| Structure PR-4 | CC30-Suc-ONa | 2 |
| | MS-275 | 0.016 |
| Structure PR-1 | Gly-MS-275•HCl | 68 |
| Structure PR-1 | Val-MS-275•HCl | >100 |
| Structure PR-1 | Lys-MS-275•2HCl | >100 |
| Structure PR-1 | Ser-MS-275•HCl | >100 |
| Structure PR-1 | Thr-MS-275•HCl | >100 |
| | MGCD0103 | <1 |
| Structure PR-2 | Gly-MGCD0103•HCl | >100 |
| Structure PR-2 | Val-MGCD0103•HCl | >100 |
| Structure PR-2 | Lys-MGCD0103•2HCl | >100 |
| Structure PR-2 | Ser-MGCD0103•HCl | >100 |
| Structure PR-2 | Thr-MGCD0103•HCl | >100 |
| | PAOA | .033 |
| Structure PR-3 | Gly-PAOA•HCl | 11 |
| Structure PR-3 | Val-PAOA•HCl | >100 |
| Structure PR-3 | Lys-PAOA•2HCl | >100 |
| Structure PR-3 | Thr-PAOA•HCl | >100 |

### Example 3

### Conversion of the Prodrug Lys-CC30.2HCl to Parent Drug CC30 In Vitro and In Vivo

I. Prodrug Lys-CC30.2HCl Converted In Vitro to the Parent Drug CC30 in Rat Plasma

Lys-CC30.2HCl (2.53 mg) was dissolved in 200 μl of water to obtain an aqueous solution concentration of 12.6 mg/ml. 4 μl of the Lys-CC30.2HCl solution was thoroughly mixed with 100 μl of rat plasma. This mixture was divided into 10 aliquots (10 μl) placed in 1.5 ml tubes. One tube was placed on ice and was used as the 0 time point. The other 9 samples were placed in an incubator at 37° C. At 5, 10, 30, 60, 120, and 240 minutes, a tube was taken out from incubation and placed on ice to stop or slow down the reaction. The samples were diluted with water (50-fold), mixed, and pelleted by centrifugation at 4° C. and 14,000 rpm for 5 min. The supernatant (˜200-300 μl) was used for HPLC-MS analysis. The % conversion was calculated by dividing the peak area of the parent drug by the peak area of the prodrug at time 0, and then multiplying by 100 (Table 10).

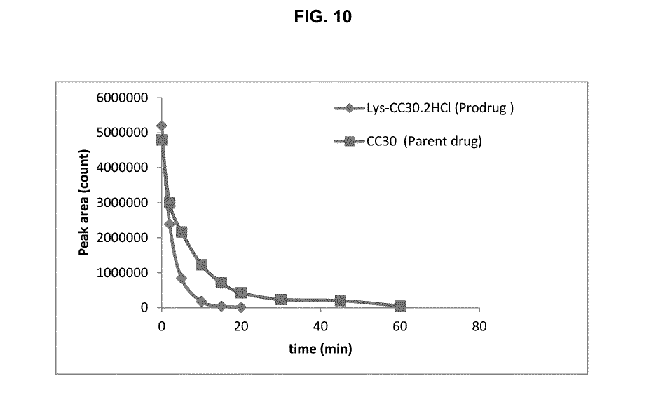

Table 10 shows the percent of Lys-CC30.2HCl hydrolyzed to CC30 in rat plasma at different time intervals between 0 and 240 minutes. FIG. 10A shows a graph of the drug concentrations in the rat plasma measured as HPLC-MS peak areas over a 240 minute time period. After 60 minutes of incubation, almost half (44.7%) of the Lys-CC30.2HCl prodrug converted to CC30 (Table 10).

TABLE 10
HPLC-MS Peak Area of Lys-CC30•2HCl (prodrug)
and CC30 (parent drug) after incubation in rat plasma

| Time | Prodrug, | Parent Drug, | % |
|---|---|---|---|
| (min) | Lys-CC30•2HCl | CC30 | Conversion |
| 0 | 6182672 | 617276 | 10.0% |
| 5 | 5900784 | 880074 | 14.2% |
| 10 | 5623041 | 1131442 | 18.3% |
| 30 | 5199304 | 2039869 | 33.0% |
| 60 | 3678701 | 2762843 | 44.7% |
| 120 | 921181 | 4280514 | 69.2% |
| 240 | 498331 | 4438887 | 71.8% |

II. Prodrug Lys-CC30.2HCl Converted In Vivo to the Parent Drug CC30 in a Rat

A Male Sprague-Dawley rat weighing 200-220 g was used to study the pharmacokinetics of the prodrug Lys-CC30.2HCl. Food was prohibited for 12 hours before the experiment, but water was freely available. Blood samples (0.2 ml) were collected through orbital venous plexus blood into heparinized 1.5 ml polyethene tubes at 0, 2, 5, 10, 15, 20, 30, 45 and 60 minute time points after the intravenous administration of Lys-CC30.2HCl (2 mg/kg). The samples were immediately centrifuged at 3000 g for 10 minutes. The plasma obtained (100 μl) was stored at −20° C. until analyzed. Plasma Lys-CC30.2HCl and parent drug CC30 concentrations in the collected blood samples were analyzed by HPLC-MS.

The data shows that the prodrug Lys-CC30.2HCl immediately started converting in the rat to the parent drug CC30 because after injection of Lys-CC30.2HCl at time zero approximately the same amount of the parent CC30 drug was detected as was the prodrug Lys-CC30.2HCl (Table 11)(FIG. 10B). The parent drug was detected in the rat for at least 60 minutes, whereas the prodrug was no longer detected after 20 minutes (Table 11)(FIG. 10B).

TABLE 11
HPLC-MS Peak Area of Lys-CC30•2HCl (prodrug) and
CC30 (parent drug) in the Rat at Different Time Points

| | Prodrug, | Parent drug, |
|---|---|---|
| Time(min) | Lys-CC30•2HCl | CC30 |
| 0 | 5199661 | 4792335 |
| 2 | 2388965 | 2994581 |
| 5 | 838012 | 2162228 |
| 10 | 173555 | 1225981 |
| 15 | 40200 | 712348 |
| 20 | 8674 | 425079 |
| 30 | | 232663 |
| 45 | | 199152 |
| 60 | | 44686 |

### Example 4

### CC30 Amide Derivatives Did not Cyclize in an Acidic Condition

Simulated gastric fluid (SGF) was prepared by thoroughly mixing diluted hydrochloric acid (16.4 ml), water (800 ml), and 10 g of pepsin together, and then bring the volume up to 1000 ml with water (pH=1.3). Diluted hydrochloric acid was prepared by mixing 234 ml of concentrated hydrochloric acid with water to a final volume of 1000 ml. Lys-CC30.2HCl (2.53 mg) or CC30 were separately dissolved in 200 μl of water to obtain an aqueous solution concentration of 12.6 mg/ml. 4 μl of the Lys-CC30.2HCl solution and the CC30 solution were thoroughly mixed with 1 ml of SGF in separate tubes. The mixtures were placed in an incubator at 37° C. for 4 hours. The samples were diluted with water 50-fold, mixed, and pelleted by centrifugation at 4° C. and 14,000 rpm for 5 min. The supernatants (˜200-300 μl) were used for HPLC-MS analysis. In acidic conditions of the SGF solution, 5% of the parent drug CC30 formed an inactive cyclized product:

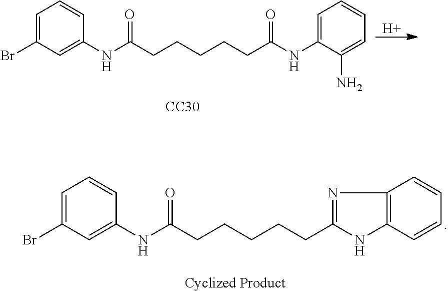

No similarly cyclized product was formed when Lys-CC30.2HCl was incubated in the SGF solution. The inability of the prodrug Lys-CC30.2HCl to form the inactive cyclized product is a new advantageous property.

### REFERENCES

The reference listed below, and all references cited in the specification above are hereby incorporated by reference in their entirety, as if fully set forth herein.

1. http://www.selleckchem.com/products/MGCD0103(Mocetinostat).html

What is claimed is:

1. A compound comprising a structure of Structure X: 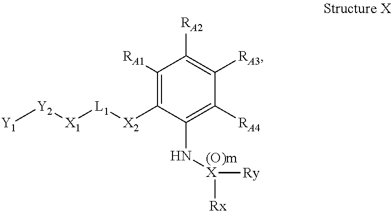
including crystals, stereoisomers, pharmaceutically acceptable solvates, and pharmaceutically acceptable salts thereof, further including mixtures thereof in all ratios, wherein:
each R_{A1}-R_{A4} are independently selected from the group consisting of H, F, Cl, Br, and I;
X₂ is selected from the group consisting of —C(═O)—NH— and —NH—C(═O)—;
L₁ is —(CH₂)ₙ—, wherein n is 1, 2, 3, 4, 5, 6, 7, 8, 9, or 10, and any one or more of the —CH₂— may be replaced by a group selected from the group consisting of aryl, heteroaryl, cycloalkyl, heterocycloalkyl, substituted aryl, substituted heteroaryl, substituted cycloalkyl, substituted heterocycloalkyl, —O—, —S—, —C(═O)—, —S(═O)—, —S(═O)₂—, —NH—C(═O)—, —C(═O)—NH—, —NR—, —C═C—, and —C≡C—;
X₁ is selected from the group consisting of —C(═O)—NH—, —NH—C(═O)—, and —NH—;
Y₁ is selected from the group consisting of H, aryl, heteroaryl, cycloalkyl, heterocycloalkyl, substituted aryl, substituted heteroaryl, substituted cycloalkyl, substituted heterocycloalkyl, —OH, —SH, and —NH₂;
Y₂ is —(CH₂)ₚ—, wherein p is 1, 2, 3, 4, 5, 6, 7, 8, 9, or 10, and any one or more of the —CH₂— may be replaced by a group selected from the group consisting of alkyl, —C═C—, —C≡C—, aryl, heteroaryl, cycloalkyl, heterocycloalkyl, substituted aryl, substituted heteroaryl, substituted cycloalkyl, substituted heterocycloalkyl, —O—, —S—, —N—, —C(═O)—, and —C(═S)—;
X is S, P or C, wherein:
when X is S, and m is 2, Rx is selected from the group consisting of H, alkyl, aryl, heteroaryl, cycloalkyl, heterocycloalkyl, substituted alkyl, substituted aryl, substituted heteroaryl, substituted cycloalkyl, and substituted heterocycloalkyl; and Ry is nothing;
when X is P, and m is 1, Rx and Ry are independently selected from the group consisting of H, alkyl, aryl, heteroaryl, cycloalkyl, heterocycloalkyl, substituted alkyl, substituted aryl, substituted heteroaryl, substituted cycloalkyl, and substituted heterocycloalkyl;
when X is C, and m is 0, Rx is H, and Ry is 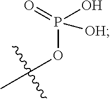 [SMILES: *OP(=O)(O)O]
and
when X is C, and m is 1, Rx is nothing, and Ry is alkyl, alkyl carboxyl, 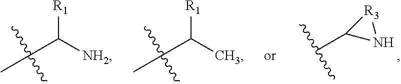
wherein
R1 selected from the group consisting of H, 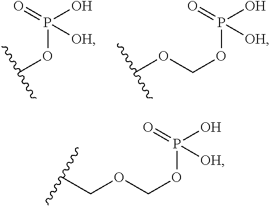 [SMILES: *OP(=O)(O)O ; *OCOP(=O)(O)O ; *COCOP(=O)(O)O]
alkyl, and alkyl further substituted with aryl, heteroaryl, amino, hydroxide, hydroxide aryl, hydroxide carbonyl, amino carbonyl, thiol, alkyl-S—, or guanidinyl; and
R3 is alkyl.

2. The compound according to claim 1, wherein 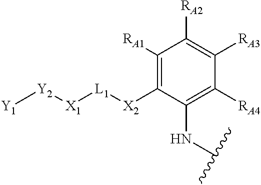
is selected from the group consisting of Structure PR-1, Structure PR-2, Structure PR-3, Structure PR-4, Structure PR-5, Structure PR-6, Structure PR-7, Structure PR-8, and Structure PR-9.

3. The compound according to claim 2, wherein
X is C; m is 0; Rx is H; and
Ry is

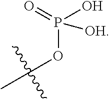 [SMILES: *OP(=O)(O)O]

4. The compound according to claim 2, wherein
X is C;
m is 1;
Rx is nothing;
Ry is selected from the group consisting of alkyl, alkyl carboxyl, 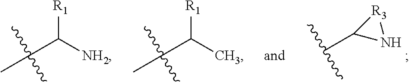
R₁ is selected from the group consisting of H, 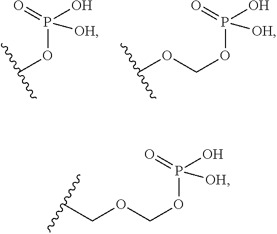 [SMILES: *OP(=O)(O)O ; *OCOP(=O)(O)O ; *COCOP(=O)(O)O]
alkyl, alkyl further substituted with aryl, heteroaryl, amino, hydroxide, hydroxide aryl, hydroxide carbonyl, amino carbonyl, thiol, alkyl-S—, or guanidinyl; and
R₃ is alkyl.

5. The compound according to claim 4, having a structure selected from the group consisting of

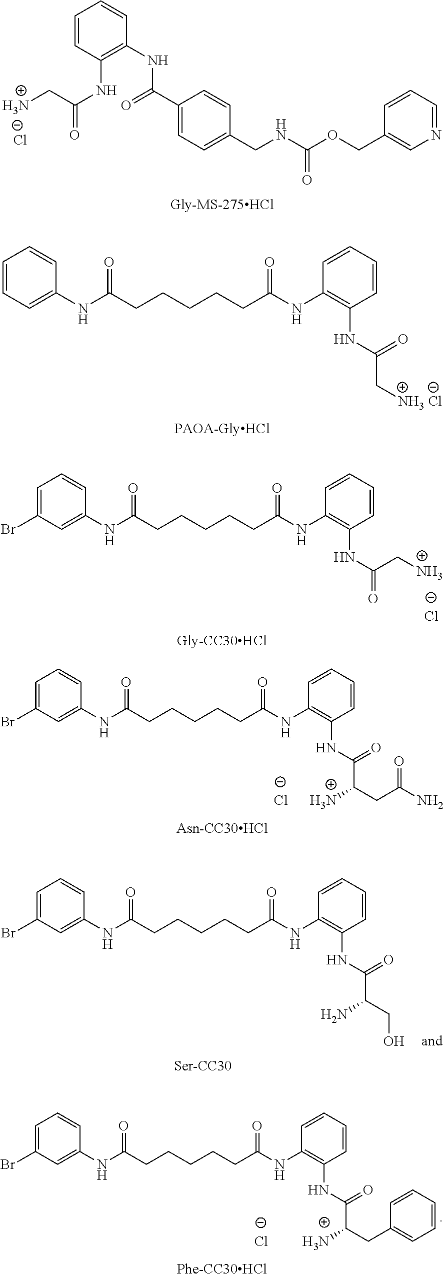

6. The compound according to claim 1, wherein 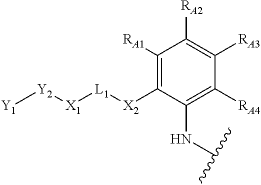
is 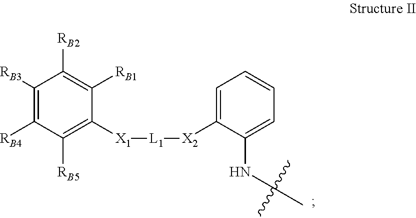
R_{B1}, R_{B2}, R_{B5}, and R_{B6} are hydrogen;
R_{B3} and R_{B4} are independently selected from the group consisting of hydrogen, haloalkyl and halogen;
X₁ and X₂ are independently selected from the group consisting of —C(═O)—NH— and —NH—C(═O)—; and
L₁ is —(CH₂)ₙ—, wherein n is 4, 5, 6, 7, or 8.

7. The compound according to claim 6, wherein
X is C;
m is 1;
Rx is nothing;
Ry is 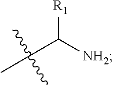
and
R₁ is selected from the group consisting of H, alkyl, and alkyl further substituted with a substituent selected from the group consisting of aryl, heteroaryl, amino, hydroxide, hydroxide aryl, hydroxide carbonyl, amino carbonyl, thiol, alkyl-S—, and guanidinyl.

9. The compound according to claim 6, wherein
X is C;
m is 0;
Rx is H; and
Ry is

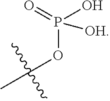 [SMILES: *OP(=O)(O)O]

10. A composition comprising the compound according to claim 1.

11. A method for using the compound according to claim 1 for a condition treatable in a subject by the corresponding 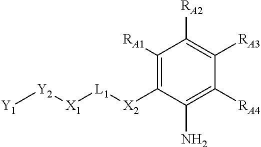
comprising:
applying to the subject a therapeutically effective amount of the compound, or the composition thereof.
